(19) United States
(12) Patent Application Publication
(10) Pub. No.: US 2023/0241211 A1
(43) Pub. Date: Aug. 3, 2023

(54) BISPECIFIC ANTIBODY AGAINST CD3 AND CD20 IN COMBINATION THERAPY FOR TREATING DIFFUSE LARGE B-CELL LYMPHOMA

(71) Applicant: GENMAB A/S, Copenhagen V (DK)

(72) Inventors: Christopher W. L. CHIU, Warren, NJ (US); Mariana Cota STIRNER, Summit, NJ (US); Minh H. DINH, Northbrook, IL (US); Iliana Edith SZAFER-GLUSMAN, San Jose, CA (US)

(21) Appl. No.: 18/160,386

(22) Filed: Jan. 27, 2023

Related U.S. Application Data
(60) Provisional application No. 63/304,424, filed on Jan. 28, 2022.

(51) Int. Cl. A61K 39/395; A61K 31/454; A61K 31/519; A61P 35/00; A61P 7/00

(52) U.S. Cl. CPC A61K 39/3955; A61K 31/454; A61K 31/519; A61P 35/00; A61P 7/00; A61K 2039/545

(57) ABSTRACT

Provided are methods of clinical treatment of Diffuse Large B-cell Lymphoma (for example, relapsed and/or refractory Diffuse Large B-cell Lymphoma) in human subjects using a bispecific antibody which binds to CD3 and CD20 in combination with lenalidomide or ibrutinib and lenalidomide.

### FIELD

The present invention relates to bispecific antibodies targeting both CD3 and CD20 and the use of such antibodies in combination with lenalidomide or in combination with lenalidomide and ibrutinib for the treatment of diffuse large B-cell lymphoma (DLBCL), for example, relapsed and/or refractory DLBCL. Advantageous treatment regimens are also provided.

### BACKGROUND

DLBCL is the most common non-Hodgkin lymphoma (NHL), and the standard first-line therapy is R-CHOP. The cure rate of this combination for the overall population of newly-diagnosed DLBCL is between 60% and 70% (Sehn et al., Blood 2007; 109:1867-61). Attempts to improve upon outcomes of first-line therapy, including intensification of dose and addition of other agents to intensify the regimen, have failed to provide sufficient evidence to alter standard of care.

Risk factors impacting rates of CR to first-line treatment, disease relapse, and OS are included in the International Prognostic Index (IPI) or Revised-IPI (R-IPI): age >60 years, ECOG>1 or KPS<60, LDH>ULN; extranodal disease >1 (2 or more), and disease Stage 3 or 4 (Project et al., N Engl J Med 1993; 329:987-994; Sehn et al., supra). While patients in the good risk group (1-2 IPI factors) have a 4-year PFS of 80% following standard first-line R-CHOP, the 45% of patients in the poor risk (high risk) group (3-5 IPI factors) only achieve a 4-year PFS and OS of 55% (Sehn et al., supra).

Given the limited efficacy and long-term response of poor risk subjects to currently available treatments, novel and effective treatments are needed.

### SUMMARY

Provided herein are methods of treating human subjects who have of diffuse large B-cell lymphoma (DLBCL), for example, relapsed and/or refractory (R/R) DLBCL, by administering a bispecific antibody which binds to CD3 and CD20 in combination with lenalidomide or in combination lenalidomide and ibrutinib, in particular, advantageous clinical treatment regimens.

In one aspect, provided herein is a method of treating DLBCL, for example, R/R DLBCL, in a human subject, the method comprising administering to the subject the combination of epcoritamab with lenalidomide or with lenalidomide and ibrutinib, e.g., the method comprising administering to the subject an effective amount of lenalidomide, and epcoritamab or ibrutinib, lenalidomide and epcoritamab.

In one aspect, provided herein is a method of treating DLBCL, for example, R/R DLBCL in a human subject, the method comprising administering to the subject a bispecific antibody (e.g. subcutaneously) and an effective amount of lenalidomide lenalidomide (e.g., orally), or an effective amount of lenalidomide (e.g. orally) and ibrutinib (e.g. orally), wherein the bispecific antibody comprises:

(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and

(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14,

wherein the bispecific antibody is administered at a dose of 24 mg or 48 mg, and wherein lenalidomide, and the bispecific antibody, and optionally ibrutinib, are administered in 28-day cycles.

In some embodiments, the bispecific antibody is administered at a dose of (or a dose of about) 24 mg. In some embodiments, the bispecific antibody is administered at a dose of (or a dose of about) 48 mg.

In one embodiment, the bispecific antibody is administered once every week at a dose of 24 mg or 48 mg (weekly administration), e.g., for 2.5 28-day cycles. In some embodiments, the bispecific antibody is administered once every four weeks after the biweekly administration, e.g., for at least eight 28-day cycles, e.g., until disease progression or unacceptable toxicity. In a further embodiment, a priming dose (e.g., 0.16 mg or about 0.16 mg) of the bispecific antibody is administered two weeks prior to administering the first weekly dose of 24 mg or 48 mg. In some embodiments, after administering the priming dose and prior to administering the weekly dose of 24 mg or 48 mg, an intermediate dose (e.g., 0.8 mg or about 0.8 mg) of the bispecific antibody is administered. In some embodiments, the priming dose is administered one week before the intermediate dose, and the intermediate dose is administered one week before the first weekly dose of 24 mg or 48 mg.

In some embodiments, ibrutinib is administered in a 28-day cycle once every day (daily administration), e.g., for up to twenty-four 28-day cycles, or for at least twenty-four 28-day cycles, such as for twenty-four 28-day cycles. In one embodiment, ibrutinib is administered at a dose of about 420 mg/day, such as 420 mg/day. In one embodiment, ibrutinib is administered at a dose of about 560 mg/day, such as 560 mg/day.

In some embodiments, lenalidomide is administered once a day from day 1 to day 21 of the 28-day cycles, e.g., from cycle 1 to cycle 12 of the 28-day cycles. In some embodiments, lenalidomide is administered at a dose of about 25 mg, such as 25 mg, in cycles 1-12 of the 28-day cycles. In some embodiments, lenalidomide is administered at a dose of about 20 mg, such as 20 mg, in cycle 1 to cycle 24 of the 28-day cycles.

In some embodiments, lenalidomide and optionally ibrutinib, and the bispecific antibody are administered on the same day (e.g., on days 1, 8, 15 and 22 of cycles 1-3, and on day 1 of cycles 4-12, or on days 1, 8, 15 and 22 of cycles 1-3, and on day 1 of cycles 4-24.

In some embodiments, administration is performed in 28-day cycles, wherein:

(a) the bispecific antibody is administered as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2 and 3, a dose of 24 mg is administered on days 1, 8, 15, and 22;
(iii) in cycle 4 and onwards, a dose of 24 mg is administered on day 1;

(b) lenalidomide is administered on days 1-21 in cycle 1 and onwards, and

(c) ibrutinib is optionally administered on days 1-28 in cycle 1 and onwards.

In some embodiments, administration is performed in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-12, a dose of 24 mg is administered on day 1; and

(b) lenalidomide is administered orally at a dose of 25 mg/day on days 1-21 in cycles 1-12.

In some embodiments, administration is performed in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-24, a dose of 24 mg is administered on day 1;

(b) lenalidomide is administered orally at a dose of 20 mg/day on days 1-21 in cycles 1-24 and

(c) ibrutinib is administered orally at a dose of 560 mg/day on days 1-28 in cycles 1-24.

In some embodiments, administration is performed in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-24, a dose of 24 mg is administered on day 1;

(b) lenalidomide is administered orally at a dose of 20 mg/day on days 1-21 in cycles 1-24 and

(c) ibrutinib is administered orally at a dose of 420 mg/day on days 1-28 in cycles 1-24.

In some embodiments, administration is performed in 28-day cycle, wherein:

(a) the bispecific antibody is administered as follows:
(i) in cycle 1, a priming dose of 0.16 mg is administered on day 1, an intermediate dose of 0.8 mg is administered on day 8, and a dose of 48 mg is administered on days 15 and 22;
(ii) in cycles 2 and 3, a dose of 48 mg is administered on days 1, 8, 15, and 22;
(iii) in cycle 3 and onwards, a dose of 48 mg is administered on day 1;

(b) lenalidomide is administered on days 1-21 in cycles 1 and onwards; and

(c) ibrutinib is optionally administered on days 1-28 in cycle 1 and onwards.

In some embodiments, administration is performed in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycles 2 and 3, a dose of 48 mg is administered on days 1, 8, 15, and 22;
(iii) in cycles 4-12, a dose of 48 mg is administered on day 1; and

(b) lenalidomide is administered orally at a dose of 25 mg/day on days 1-21 in cycles 1-12.

In some embodiments, administration is performed in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycle 2 and 3, a dose of 48 mg is administered on days 1, 8, 15, and 22;
(iii) in cycles 4-24, a dose of 48 mg is administered on day 1;

(b) lenalidomide is administered orally at a dose of 20 mg/day on days 1-21 in cycles 1-24 and

(c) ibrutinib is administered orally at a dose of 560 mg/day on days 1-28 in cycles 1-24.

In some embodiments, administration is performed in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycle 2 and 3, a dose of 48 mg is administered on days 1, 8, 15, and 22;
(iii) in cycles 4-24, a dose of 48 mg is administered on day 1;

(b) lenalidomide is administered orally at a dose of 20 mg/day on days 1-21 in cycles 1-24 and

(c) ibrutinib is administered orally at a dose of 420 mg/day on days 1-28 in cycles 1-24.

In some embodiments, the bispecific antibody is administered subcutaneously. In some embodiments, ibrutinib is administered orally. In some embodiments, lenalidomide is administered orally.

In some embodiments, the DLBCL is with histologically confirmed CD20+ disease. In some embodiments, the DLBCL is high-grade B cell lymphoma with MYC and BCL-2 and/or BCL-6 translocations (double-hit or triple-hit). In some embodiments, the DLBCL is follicular lymphoma Grade 3B. In some embodiments the DLBCL is relapsed and/or refractory DLBCL.

In some embodiments, the first antigen-binding region of the bispecific antibody comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and the second antigen-binding region comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively. In some embodiments, the first antigen-binding region of the bispecific antibody comprises a VH region comprising the amino acid sequence of SEQ ID NO: 6, and the VL region comprising the amino acid sequence of SEQ ID NO: 7; and the second antigen-binding region comprises a VH region comprising the amino acid sequence of SEQ ID NO: 13, and the VL region comprising the amino acid sequence of SEQ ID NO: 14.

In some embodiments, the first binding arm of the bispecific antibody is derived from a humanized antibody, preferably from a full-length IgG1,λ (lambda) antibody (e.g., SEQ ID NO: 22). In some embodiments, the second binding arm of the bispecific antibody is derived from a human antibody, preferably from a full-length IgG1,κ (kappa) antibody (e.g., SEQ ID NO: 23).

In some embodiments, the bispecific antibody is a full-length antibody with a human IgG1 constant region.

In some embodiments, the bispecific antibody comprises an inert Fc region, for example, an Fc region in which the amino acids in the positions corresponding to positions L234, L235, and D265 in the human IgG1 heavy chain constant region of SEQ ID NO: 15 are F, E, and A, respectively. In some embodiments, the bispecific antibody comprises substitutions which promote bispecific antibody formation, for example, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa. In some embodiments, the bispecific antibody has both an inert Fc region (e.g., substitutions at L234, L235, and D265 (e.g., L234F, L235E, and D265A)) and substitutions which promote bispecific antibody formation (e.g., F405L and K409R). In a further embodiment, the bispecific antibody comprises heavy chain constant regions comprising the amino acid sequences of SEQ ID NOs: 19 and 20.

In some embodiments, the bispecific antibody comprises a first heavy chain and a first light chain comprising (or consisting of) the amino acid sequences set forth in SEQ ID NOs: 24 and 25, respectively, and a second heavy chain and a second light chain comprising (or consisting of) the amino acid sequences set forth in SEQ ID NOs: 26 and 27, respectively. In some embodiments, the bispecific antibody is epcoritamab, or a biosimilar thereof.

### BRIEF DESCRIPTION OF THE DRAWINGS

FIG. 1 shows T-cell activation in the presence of lenalidomide. T cells were pre-incubated with assay medium or lenalidomide in the absence or presence of CD3 crosslinking by immobilized anti-CD3 for 3 days (as indicated on the x-axes), after which upregulation of CD69, CD25, PD-1 and LAMP-1 on CD4⁺ and CD8⁺ T cells was determined by flow cytometry. Data shown are geomean fluorescence intensities (FI) for four donors (each circle represents one condition for one donor). Pre-incubation conditions are shown on the x-axis.

FIG. 2 is a graph showing the effects of lenalidomide on Duobody®-CD3×CD20-induced T-cell-mediated cytotoxicity against CD20-expressing Daudi cells. T cells were incubated with (5 or 50 μM) or without lenalidomide in the absence or presence of immobilized anti-CD3 for 3 days. T cells were then used in a cytotoxicity assay with DuoBody®-CD3×CD20 or DuoBody®-CD3×ctrl (containing a CD3 arm and a non-binding control arm) and CD20-expressing Daudi cells as target cells (E:T ratio 2:1). Data shown are average percentages cytotoxicity ±SD of duplicates, normalized to medium control (no antibody, no lenalidomide).

FIG. 3 is a schematic of the overall clinical trial design.

### DETAILED DESCRIPTION

### Definitions

The term “immunoglobulin” as used herein refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized (see, e.g., Fundamental Immunology Ch. 7 (Paul, W., ed., 2nd ed. Raven Press, N.Y. (1989)). Briefly, each heavy chain typically is comprised of a heavy chain variable region (abbreviated herein as VH or V_{H}) and a heavy chain constant region (abbreviated herein as CH or C_{H}). The heavy chain constant region typically is comprised of three domains, CH1, CH2, and CH3. The hinge region is the region between the CH1 and CH2 domains of the heavy chain and is highly flexible. Disulfide bonds in the hinge region are part of the interactions between two heavy chains in an IgG molecule. Each light chain typically is comprised of a light chain variable region (abbreviated herein as VL or VL) and a light chain constant region (abbreviated herein as CL or C_{L}). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J Mol Biol 1987; 196:901-17). Unless otherwise stated or contradicted by context, CDR sequences herein are identified according to IMGT rules (Brochet X., Nucl Acids Res 2008; 36:W503-508; Lefranc M P., Nucl Acids Res 1999; 27:209-12; www.imgt.org/). Unless otherwise stated or contradicted by context, reference to amino acid positions in the constant regions is according to the EU-numbering (Edelman et al., PNAS. 1969; 63:78-85; Kabat et al., Sequences of Proteins of Immunological Interest, Fifth Edition. 1991 NIH Publication No. 91-3242). For example, SEQ ID NO: 15 sets forth amino acids positions 118-447, according to EU numbering, of the IgG1 heavy chain constant region.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is within the ability of one of ordinary skill in the art to align a sequence or segment in a sequence and thereby determine the corresponding position in a sequence to an amino acid position according to the present invention.

The term “antibody” (Ab) as used herein in the context of the present invention refers to an immunoglobulin molecule which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term antibody, unless specified otherwise, also encompasses polyclonal antibodies, monoclonal antibodies (mAbs), antibody-like polypeptides, chimeric antibodies and humanized antibodies

The term “antibody fragment” or “antigen-binding fragment” as used herein refers to a fragment of an immunoglobulin molecule which retains the ability to specifically bind to an antigen, and can be generated by any known technique, such as enzymatic cleavage, peptide synthesis, and recombinant techniques. Examples of antibody fragments include (i) a Fab′ or Fab fragment, a monovalent fragment consisting of the VL, VH, CL and CH1 domains, or a monovalent antibody as described in WO2007059782 (Genmab); (ii) F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 1989; 341: 544-46), which consists essentially of a VH domain and also called domain antibodies (Holt et al; Trends Biotechnol 2003; 21:484-90); (vi) camelid or nanobodies (Revets et al; Expert Opin Biol Ther 2005; 5:111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see, e.g., Bird et al., Science 1988; 242:423-26 and Huston et al., PNAS 1988; 85:5879-83). Such single chain antibodies are encompassed within the term antibody fragment unless otherwise noted or clearly indicated by context.

The term “antibody-binding region” or “antigen-binding region” as used herein refers to the region which interacts with the antigen and comprises both the VH and the VL regions. The term antibody when used herein refers not only to monospecific antibodies, but also multispecific antibodies which comprise multiple, such as two or more, e.g., three or more, different antigen-binding regions. The term antigen-binding region, unless otherwise stated or clearly contradicted by context, includes fragments of an antibody that are antigen-binding fragments, i.e., retain the ability to specifically bind to the antigen.

As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g., IgG1, is mentioned, the term is not limited to a specific isotype sequence, e.g., a particular IgG1 sequence, but is used to indicate that the antibody is closer in sequence to that isotype, e.g. IgG1, than to other isotypes. Thus, e.g., an IgG1 antibody may be a sequence variant of a naturally-occurring IgG1 antibody, which may include variations in the constant regions.

The term “bispecific antibody” or “bs” or “bsAb” as used herein refers to an antibody having two different antigen-binding regions defined by different antibody sequences. A bispecific antibody can be of any format.

The terms “half molecule”, “Fab-arm”, and “arm”, as used herein, refer to one heavy chain-light chain pair.

When a bispecific antibody is described as comprising a half-molecule antibody “derived from” a first parental antibody, and a half-molecule antibody “derived from” a second parental antibody, the term “derived from” indicates that the bispecific antibody was generated by recombining, by any known method, said half-molecules from each of said first and second parental antibodies into the resulting bispecific antibody. In this context, “recombining” is not intended to be limited by any particular method of recombining and thus includes all of the methods for producing bispecific antibodies described herein, including for example recombining by half-molecule exchange (also known as “controlled Fab-arm exchange”), as well as recombining at nucleic acid level and/or through co-expression of two half-molecules in the same cells.

The term “full-length” as used herein in the context of an antibody indicates that the antibody is not a fragment but contains all of the domains of the particular isotype normally found for that isotype in nature, e.g., the VH, CH1, CH2, CH3, hinge, VL and CL domains for an IgG1 antibody. A full-length antibody may be engineered. An example of a “full-length” antibody is epcoritamab.

The term “Fc region” as used herein refers to an antibody region consisting of the Fc sequences of the two heavy chains of an immunoglobulin, wherein said Fc sequences comprise at least a hinge region, a CH2 domain, and a CH3 domain.

The term “heterodimeric interaction between the first and second CH3 regions” as used herein refers to the interaction between the first CH3 region and the second CH3 region in a first-CH3/second-CH3 heterodimeric protein.

The term “homodimeric interactions of the first and second CH3 regions” as used herein refers to the interaction between a first CH3 region and another first CH3 region in a first-CH3/first-CH3 homodimeric protein and the interaction between a second CH3 region and another second CH3 region in a second-CH3/second-CH3 homodimeric protein.

The term “binding” as used herein in the context of the binding of an antibody to a predetermined antigen typically refers to binding with an affinity corresponding to a K_{D} of about 10⁻⁶ M or less, e.g., 10⁻⁷ M or less, such as about 10⁻⁸ M or less, such as about 10⁻⁹ M or less, about 10⁻¹⁰ M or less, or about 10⁻¹¹M or even less, when determined by, e.g., BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (i.e., the antibody is highly specific).

The term “isolated antibody” as used herein refers to an antibody which is substantially free of other antibodies having different antigenic specificities. In a preferred embodiment, an isolated bispecific antibody that specifically binds to CD20 and CD3 is in addition substantially free of monospecific antibodies that specifically bind to CD20 or CD3.

The term “CD3” as used herein refers to the human Cluster of Differentiation 3 protein which is part of the T-cell co-receptor protein complex and is composed of four distinct chains. CD3 is also found in other species, and thus, the term “CD3” is not limited to human CD3 unless contradicted by context. In mammals, the complex contains a CD3γ (gamma) chain (human CD3γ chain UniProtKB/Swiss-Prot No P09693, or cynomolgus monkey CD3γ UniProtKB/Swiss-Prot No Q95LI7), a CD3δ (delta) chain (human CD3δ UniProtKB/Swiss-Prot No P04234, or cynomolgus monkey CD3δ UniProtKB/Swiss-Prot No Q95LI8), two CD3ε (epsilon) chains (human CD3ε UniProtKB/Swiss-Prot No P07766, SEQ ID NO: 28); cynomolgus CD3ε UniProtKB/Swiss-Prot No Q95LI5; or rhesus CD3ε UniProtKB/Swiss-Prot No G7NCB9), and a CD3ζ-chain (zeta) chain (human CD3ζ UniProtKB/Swiss-Prot No P20963, cynomolgus monkey CD3ζ UniProtKB/Swiss-Prot No Q09TK0). These chains associate with a molecule known as the T-cell receptor (TCR) and generate an activation signal in T lymphocytes. The TCR and CD3 molecules together comprise the TCR complex.

The term “CD3 antibody” or “anti-CD3 antibody” as used herein refers to an antibody which binds specifically to the antigen CD3, in particular human CD3ε (epsilon).

The term “human CD20” or “CD20” refers to human CD20 (UniProtKB/Swiss-Prot No P11836, SEQ ID NO: 29) and includes any variants, isoforms, and species homologs of CD20 which are naturally expressed by cells, including tumor cells, or are expressed on cells transfected with the CD20 gene or cDNA. Species homologs include rhesus monkey CD20 (Macaca mulatta; UniProtKB/Swiss-Prot No H9YXP1) and cynomolgus monkey CD20 (Macaca fascicularis; UniProtKB No G7PQ03).

The term “CD20 antibody” or “anti-CD20 antibody” as used herein refers to an antibody which binds specifically to the antigen CD20, in particular to human CD20.

The term “CD3×CD20 antibody”, “anti-CD3×CD20 antibody”, “CD20×CD3 antibody” or “anti-CD20×CD3 antibody” as used herein refers to a bispecific antibody which comprises two different antigen-binding regions, one of which binds specifically to the antigen CD20 and one of which binds specifically to CD3.

The term “DuoBody®-CD3×CD20” as used herein refers to an IgG1 bispecific CD3×CD20 antibody comprising a first heavy and light chain pair as defined in SEQ ID NO: 24 and SEQ ID NO: 25, respectively, and comprising a second heavy and light chain pair as defined in SEQ ID NO: 26 and SEQ ID NO: 27. The first heavy and light chain pair comprises a region which binds to human CD3ε (epsilon), the second heavy and light chain pair comprises a region which binds to human CD20. The first binding region comprises the VH and VL sequences as defined by SEQ ID NOs: 6 and 7, and the second binding region comprises the VH and VL sequences as defined by SEQ ID NOs: 13 and 14. This bispecific antibody can be prepared as described in WO 2016/110576.

Antibodies comprising functional variants of the heavy chain, light chains, VL regions, VH regions, or one or more CDRs of the antibodies of the examples as also provided herein. A functional variant of a heavy chain, a light chain, VL, VH, or CDRs used in the context of an antibody still allows the antibody to retain at least a substantial proportion (at least about 90%, 95% or more) of functional features of the “reference” and/or “parent” antibody, including affinity and/or the specificity/selectivity for particular epitopes of CD20 and/or CD3, Fc inertness and PK parameters such as half-life, Tmax, Cmax. Such functional variants typically retain significant sequence identity to the parent antibody and/or have substantially similar length of heavy and light chains. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J Mol Biol 1970; 48:444-453 algorithm. Exemplary variants include those which differ from heavy and/or light chains, VH and/or VL, and/or CDR regions of the parent antibody sequences mainly by conservative substitutions; e.g., 10, such as 9, 8, 7, 6, 5, 4, 3, 2 or 1 of the substitutions in the variant may be conservative amino acid residue replacements.

Conservative substitutions may be defined by substitutions within the classes of amino acids reflected in the following table:

TABLE 1
Amino add residue classes for conservative substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Unless otherwise indicated, the following nomenclature is used to describe a mutation: i) substitution of an amino acid in a given position is written as, e.g., K409R which means a substitution of a Lysine in position 409 with an Arginine; and ii) for specific variants the specific three or one letter codes are used, including the codes Xaa and X to indicate any amino acid residue. Thus, the substitution of Lysine with Arginine in position 409 is designated as: K409R, and the substitution of Lysine with any amino acid residue in position 409 is designated as K409X. In case of deletion of Lysine in position 409 it is indicated by K409*.

The term “humanized antibody” as used herein refers to a genetically engineered non-human antibody, which contains human antibody constant domains and non-human variable domains modified to contain a high level of sequence homology to human variable domains. This can be achieved by grafting of the six non-human antibody CDRs, which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e., the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. The VH and VL of the CD3 arm that is used herein in DuoBody®-CD3×CD20 represents a humanized antigen-binding region. Optionally, additional amino acid modifications, which are not necessarily back-mutations, may be applied to obtain a humanized antibody with preferred characteristics, such as affinity and biochemical properties.

The term “human antibody” as used herein refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The VH and VL of the CD20 arm that is used in DuoBody®-CD3×CD20 represents a human antigen-binding region. Human monoclonal antibodies of the invention can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known. Human monoclonal antibodies can thus be generated using, e.g., transgenic or transchromosomal mice or rats carrying parts of the human immune system rather than the mouse or rat system. Accordingly, in one embodiment, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogenous animal antibody machinery (see, e.g., Mendez et al. Nat Genet 1997; 15:146-56). The VH and VL regions of the CD20 arm that is used in DuoBody®-CD3×CD20 represents a human antigen-binding region.

The term “biosimilar” (e.g., of an approved reference product/biological drug) as used herein refers to a biologic product that is similar to the reference product based on data from (a) analytical studies demonstrating that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product). In some embodiments, the biosimilar biological product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. A biosimilar can be, e.g., a presently known antibody having the same primary amino acid sequence as a marketed antibody, but may be made in different cell types or by different production, purification, or formulation methods.

The term “reducing conditions” or “reducing environment” as used herein refers to a condition or an environment in which a substrate, here a cysteine residue in the hinge region of an antibody, is more likely to become reduced than oxidized.

The term “recombinant host cell” (or simply “host cell”) as used herein is intended to refer to a cell into which an expression vector has been introduced, e.g., an expression vector encoding an antibody described herein. Recombinant host cells include, for example, transfectomas, such as CHO, CHO-S, HEK, HEK293, HEK-293F, Expi293F, PER.C6 or NS0 cells, and lymphocytic cells.

The term “diffuse large B-cell lymphoma” or “DLBCL” as used herein refers to a neoplasm of the germinal center B lymphocytes with a diffuse growth pattern and a high-intermediate proliferation index. DLBCL represents approximately 30% of all lymphomas. Subtypes of DLBCL seem to have different outlooks (prognoses) and responses to treatment. DLBCL can affect any age group but occurs mostly in older people (the average age is mid-60s). “Double hit” and “triple hit” DLBCL refers to DLBCL with MYC and BCL2 and/or BCL6 translocations, falling under the category of high-grade B cell lymphoma (HGBCL) with MYC and BCL2 and/or BCL6 translocations, in accordance with the WHO 2016 classification (Swerdlow S H, Campo E, Harris N L, et al. WHO Classification of Tumours of Haematopoietic and Lymphoid Tissues (Revised ed. 4th). Lyon, France: IARC Press (2017), the contents of which are herein incorporated by reference). Follicular lymphoma grade 3B is also often considered to be equivalent to DLBCL and thus treated as such.

The term “relapsed diffuse large B-cell lymphoma” or “relapsed DLBCL” as used herein refers to diffuse large B-cell lymphoma which previously responded to therapy but progressed ≥6 months after completion of therapy.

The term “refractory diffuse large B-cell lymphoma” or “refractory DLBCL” as used herein refers to diffuse large B-cell lymphoma which either progressed during therapy, failed to achieve an objective response to prior therapy, or progressed within 6 months after completion of therapy (including maintenance therapy. The term “R/R diffuse large B-cell lymphoma” or “R/R DLBCL” as used herein, unless specified otherwise, is intended to refer to relapsed and/or refractory diffuse large B-cell lymphoma.

The term “ibrutinib” as used herein refers to an orally bioavailable, small-molecule inhibitor of Bruton's tyrosine kinase (BTK) with potential antineoplastic activity, having the chemical formula C₂₅—H₂₄—N₆—O₂ and chemical name: 1-((3R)-3-(4-amino-3-(4-phenoxyphenyl)-1H-pyrazolo(3,4-d)pyrimidin-1-yl)piperidin-1-yl)prop-2-en-1-one (Chemical Abstracts Service No. 936563-96-1). Ibrutinib is available, e.g., under the brand name Imbruvica®. The term “ibrutinib” is also intended to encompass branded and generic versions (generic equivalents) of ibrutinib, as well as pharmaceutically acceptable salts, isomers, racemates, solvates, complexes and hydrates, anhydrate forms thereof, and any polymorphic or amorphous forms thereof or combinations thereof.

The term “lenalidomide” as used herein refers to a thalidomide derivative having the chemical formula C₁₃H₁₃N₃O₃ and chemical name: 3-(4-Amino-1-oxo-1,3-dihydro-2H-isoindol-2-yl) piperidine-2,6-dione (Chemical Abstracts Service No. 191732-72-6). Lenalidomide is available, e.g., under the brand name Revlimid®. The term “lenalidomide” is also intended to encompass branded and generic versions (generic equivalents) of lenalidomide, as well as pharmaceutically acceptable salts, isomers, racemates, solvates, complexes and hydrates, anhydrate forms thereof, and any polymorphic or amorphous forms thereof or combinations thereof.

The term “treatment” refers to the administration of an effective amount of a therapeutically active antibody described herein for the purpose of easing, ameliorating, arresting or eradicating (curing) symptoms or disease states such as DLBCL. Treatment may result in a complete response (CR), partial response (PR), or stable disease (SD), for example, as defined by Lugano criteria and/or LYRIC. Treatment may be continued, for example, up to disease progression or unacceptable toxicity.

The term “administering” or “administration” as used herein refers to the physical introduction of a composition (or formulation) comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a therapeutic agent described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods. In the methods described herein, the bispecific antibody (e.g., epcoritamab) is administered subcutaneously. Other agents used in combination with the bispecific antibody, such as for cytokine release syndrome prophylaxis and/or tumor lysis syndrome (TLS) prophylaxis, may be administered via other routes, such as intravenously or orally.

The term “effective amount” or “therapeutically effective amount” refers to an amount effective, at dosages and for periods of time necessary, to achieve a desired therapeutic result. For example, dosages as defined herein for the bispecific antibody (e.g., epcoritamab), i.e., 24 mg or 48 mg, administered subcutaneously can be defined as such an “effective amount” or “therapeutically effective amount”. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects. In some embodiments, patients treated with the methods described herein will show an improvement in ECOG performance status. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or disorder (e.g., cytokine release syndrome) or of suffering a recurrence of disease, inhibits the development or recurrence of the disease.

The term “inhibits growth” of a tumor as used herein includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%.

The term “subject” as used herein refers to a human patient, for example, a human patient with Diffuse Large B-cell Lymphoma. The terms “subject” and “patient” are used interchangeably herein.

The term “buffer” as used herein denotes a pharmaceutically acceptable buffer. The term “buffer” encompasses those agents which maintain the pH value of a solution, e.g., in an acceptable range and includes, but is not limited to, acetate, histidine, TRIS® (tris (hydroxymethyl) aminomethane), citrate, succinate, glycolate and the like. Generally, the “buffer” as used herein has a pKa and buffering capacity suitable for the pH range of about 5 to about 6, preferably of about 5.5.

“Disease progression” or “PD” as used herein refers to a situation in which one or more indices of Diffuse Large B-Cell Lymphoma show that the disease is advancing despite treatment. In one embodiment, disease progression is defined based on the Lugano Response Criteria for Malignant Lymphoma (“Lugano criteria”) and/or Lymphoma Response to Immunomodulatory Therapy Criteria (LYRIC). Details regarding the Lugano criteria/classification system, including definitions for complete response (CR), partial response (PR), no response/stable disease (NR. SD), and progressive disease (PD) are provided in Cheson et al. J Clin Oncol 2014; 32:3059-68, the contents of which are incorporated by reference herein (see, in particular, Table 3 in Cheson et al., 2014). Further details regarding the Lugano criteria/classification system, are provided in Table 3.

A “surfactant” as used herein is a compound that is typically used in pharmaceutical formulations to prevent drug adsorption to surfaces and or aggregation. Furthermore, surfactants lower the surface tension (or interfacial tension) between two liquids or between a liquid and a solid. For example, an exemplary surfactant can significantly lower the surface tension when present at very low concentrations (e.g., 5% w/v or less, such as 3% w/v or less, such as 1% w/v or less such as 0.4% w/v or less, such as below 0.1% w/v or less, such as 0.04% w/v). Surfactants are amphiphilic, which means they are usually composed of both hydrophilic and hydrophobic or lipophilic groups, thus being capable of forming micelles or similar self-assembled structures in aqueous solutions. Known surfactants for pharmaceutical use include glycerol monooleate, benzethonium chloride, sodium docusate, phospholipids, polyethylene alkyl ethers, sodium lauryl sulfate and tricaprylin (anionic surfactants); benzalkonium chloride, citrimide, cetylpyridinium chloride and phospholipids (cationic surfactants); and alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl hydroxystearate, polyoxylglycerides, polysorbates such as polysorbate 20 or polysorbate 80, propylene glycol dilaurate, propylene glycol monolaurate, sorbitan esters sucrose palmitate, sucrose stearate, tricaprylin and TPGS (Nonionic and zwitterionic surfactants).

A “diluent” as used herein is one which is pharmaceutically acceptable (safe and non-toxic for administration to a human) and is useful for the preparation of dilutions of the pharmaceutical composition or pharmaceutical formulation (the terms “composition” and “formulation” are used interchangeably herein). Preferably, such dilutions of the composition dilute only the antibody concentration but not the buffer and stabilizer. Accordingly, in one embodiment, the diluent contains the same concentrations of the buffer and stabilizer as is present in the pharmaceutical composition of the invention. Further exemplary diluents include sterile water, bacteriostatic water for injection (BWFI), a pH buffered solution which is preferably an acetate buffer, sterile saline solution such as water for injection, Ringer's solution or dextrose solution. In one embodiment the diluent comprises or consists essentially of acetate buffer and sorbitol.

As used herein, the term “about” refers to a value that is no more than 10% above and no more than 10% below a specified value.

### Diffuse Large B-Cell Lymphoma Treatment Regimens

Provided herein are methods of treating Diffuse large B-cell lymphoma (DLBCL) in a human subject using a bispecific antibody which binds to CD3 and CD20 (“anti-CD3×CD20 antibody”), e.g., an isolated anti-CD3×CD20 antibody which binds to human CD3 and human CD20, in combination with lenalidomide or in combination with lenalidomide and ibrutinib. The methods are also useful for treating, e.g., relapsed and/or refractory Diffuse large B-cell lymphoma (R/R Diffuse large B-cell lymphoma). It is understood that the methods of treating Diffuse large B-cell lymphoma (e.g., R/R Diffuse large B-cell lymphoma) with a bispecific antibody which binds to both CD3 and CD20 described herein also encompass corresponding uses of the bispecific antibody for treating Diffuse large B-cell lymphoma (e.g., R/R Diffuse large B-cell lymphoma) in a human subject.

Accordingly, in one aspect, provided herein is a method of treating Diffuse large B-cell lymphoma in a human subject, the method comprising administering a bispecific antibody and an effective amount of lenalidomide (e.g., orally) a bispecific antibody and an effective amount of ibrutinib (e.g., orally) and lenalidomide (e.g., orally), wherein the bispecific antibody comprises:

(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and

(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;

wherein the bispecific antibody is administered at a dose of (or a dose of about) 24 mg or 48 mg, and wherein lenalidomide, or lenalidomide and ibrutinib, and the bispecific antibody are administered in 28-day cycles.

In some embodiments, the bispecific antibody is a full length antibody. In some embodiments, the bispecific antibody is an antibody with an inert Fc region. In some embodiments, the bispecific antibody is a full length antibody with an inert Fc region.

In some embodiments, the bispecific antibody is administered at a dose of (or a dose of about) 24 mg. In some embodiments, the bispecific antibody is administered at a dose of (or a dose of about) 48 mg.

With regard to the dose of (or dose of about) 24 mg or 48 mg of the bispecific antibody that is to be administered, or any other specified dose, it is understood that this amount refers to the amount of a bispecific antibody representing a full-length antibody, such as epcoritamab as defined in the Examples section. Hence, one may refer to administering a dose of a bispecific antibody of 24 mg as administering a dose of a bispecific antibody described herein, wherein the dose corresponds to a dose of 24 mg of epcoritamab. One of ordinary skill in the art can readily determine the amount of antibody to be administered when, for example, the antibody used differs substantially in molecular weight from the molecular weight of a full-length antibody such as epcoritamab. For instance, the amount of antibody can be calculated by dividing the molecular weight of the antibody by the weight of a full-length antibody such as epcoritamab and multiplying the outcome thereof with the specified dose as described herein. As long as the bispecific antibody (e.g., a functional variant of DuoBody® CD3×CD20) has highly similar features as DuoBody® CD3×CD20, with regard to plasma half-life, Fc inertness, and/or binding characteristics for CD3 and CD20, i.e., with regard to CDRs and epitope binding features, such antibodies are suitable for use in the methods provided herein at a dose described for a full-length antibody such as epcoritamab.

In some embodiments, the dose of bispecific antibody is administered once every week (weekly administration) in 28-day cycles. In one embodiment, the weekly administration of 24 or 48 mg is performed for 2.5 28-day cycles (i.e., 10 times). In one embodiment, the weekly dose of 24 mg or 48 mg is administered for 2.5 28-day cycles, on days 15 and 22 of cycle 1, and days 1, 8, 15, and 22 of cycles 2 and 3. In some embodiments, after the weekly administration, one may reduce the interval of administration to once every four weeks. In one embodiment, the administration once every four weeks may be performed for an extended period, for example, for at least 1 cycle, at least 2 cycles, at least 3 cycles, at least 4 cycles, at least 5 cycles, at least 6 cycles, at least 7 cycles, at least 8 cycles, at least 9 cycles, at least 10 cycles, at least 15 cycles, at least 20 cycles, or between 1-20 cycles, 1-15 cycles, 1-10 cycles, 1-5 cycles, 5-20 cycles, 5-15 cycles, or 5-10 cycles of the 28-day cycles. In a preferred embodiment, the administration once every four weeks is performed for up to eight 28-day cycles, such as for eight 28-day cycles or nine 28-day cycles. In another preferred embodiment, the administration once every four weeks is performed for up to twenty 28-day cycles, such as for twenty 28-day cycles or twenty one 28-day cycles.

In one embodiment, the weekly dose of the bispecific antibody is administered in 28-day cycles on cycles 1-3 (which may include priming and intermediate doses, as described below), and the dose once every four weeks is administered from cycle 4 onwards, for example, on cycles 4-12, or cycles 4-24 or until disease progression or unacceptable toxicity is observed in the subject.

It is understood that the doses referred to herein may also be referred to as a full or a flat dose in the scenarios above wherein, e.g., the weekly dose, and/or the dose every four weeks is administered is at the same level. Accordingly, when a dose of 48 mg is selected, preferably, at each weekly administration, and each administration every four weeks, the same dose of 48 mg is administered. Prior to administering the dose, a priming or a priming and subsequent intermediate (second priming) dose may be administered. This may be advantageous as it may help mitigate cytokine release syndrome (CRS) risk and severity, a side-effect that can occur during treatment with the bispecific anti-CD3×CD20 antibody described herein. Such priming, or priming and intermediate doses, are at a lower dose as compared with the flat or full dose.

Accordingly, in some embodiments, prior to administering the weekly dose of 24 mg or 48 mg, a priming dose of the bispecific antibody may be administered in cycle 1 of the 28-day cycles. In one embodiment, the priming dose is administered two weeks prior to administering the first weekly dose of 24 mg or 48 mg in cycle 1. In one embodiment, the priming dose is 0.16 mg (or about 0.16 mg) of the full-length bispecific antibody.

In some embodiments, after administering the priming dose and prior to administering the weekly dose of 24 mg or 48 mg, an intermediate dose of said bispecific antibody is administered. In one embodiment, the priming dose is administered one week before the intermediate dose (i.e., on day 1 of cycle 1), and the intermediate dose is administered one week before the first dose of the weekly dose of 24 mg or 48 mg (i.e., on day 8 of cycle 1). In one embodiment, the intermediate dose is 800 μg (0.8 mg) or about 800 μg (0.8 mg) of the full-length bispecific antibody.

The methods described herein involve treating human subjects who have Diffuse large B-cell lymphoma (e.g., R/R Diffuse large B-cell lymphoma) with a bispecific antibody which binds to CD3 and CD20 in combination with a regimen of lenalidomide or lenalidomide or ibrutinib.

In some embodiments, lenalidomide, or ibrutinib and lenalidomide, are administered at dosages as supported by clinical studies, according to local guidelines, and/or according to relevant local labels.

In some embodiments, ibrutinib is administered according to the product label or summary of product characteristics (see, e.g., IMBRUVICA® (ibrutinib) prescribing information, available at https://www.accessdata.fda.gov/drugsatfda_docs/label/2016/205552s007lbl.pdf). In some embodiments, ibrutinib is administered at a dose of (or a dose of about) 420 mg. In other embodiments, ibrutinib is administered at a dose of (or a dose of about) 560 mg. In some embodiments, a biosimilar of ibrutinib is used in place of ibrutinib in the methods described herein.

In some embodiments, lenalidomide is administered according to the product label or summary of product characteristics (see, e.g., REVLIMID® prescribing information, available at www.accessdata.fda.gov/drugsatfda_docs/label/2013/021880s034lbl.pdf).

In one embodiment, ibrutinib is administered once every day (daily administration; 7QW) in 28-day cycles. In one embodiment, the daily administration of ibrutinib is performed for at least one 28-day cycle (i.e., 4 times), such as at least ten 28-day cycles, such as at least twenty 28-day cycles, such as twenty-four 28-day cycles. In one embodiment, lenalidomide is administered according to local guidelines and local labels. In some embodiments, lenalidomide is administered at a dose of (or a dose of about) 10 mg oto 25 mg. In some embodiments, lenalidomide is administered at a dose of (or a dose of about) 20 mg to 30 mg. In one embodiment, lenalidomide is administered at a dose of (or a dose of about) 20 mg. In one embodiment, lenalidomide is administered at a dose of (or a dose of about) 25 mg. In one embodiment, lenalidomide is administered as an oral dose. In one embodiment, lenalidomide is administered as a capsule for oral administration.

In one embodiment, lenalidomide is administered for 21 consecutive days (i.e., days 1-21) in 28-day cycles i.e. once a day from day 1 to day 21 of the 28-day cycles. In one embodiment, lenalidomide is administered for at least one 28-day cycle, such as least five 28-day cycles, at least ten 28-day cycles, at least fifteen 28-day cycles, at least twenty 28-day cycles or at least twenty-four 28-day cycles. In one embodiment, lenalidomide is administered for up to twelve, such as for twelve 28-day cycles (i.e., on days 1-21 of cycles 1-12 of the 28-day cycles).

In one embodiment, lenalidomide is administered for up to twenty-four 28-day cycles, such as for twenty-four 28-day cycles (i.e., on days 1-21 of cycles 1-24 of the 28-day cycles). In one embodiment, lenalidomide is administered on days 1-21 of cycles 1-12 of the 28-day cycles at a dose of (or a dose of about) 25 mg. In one embodiment, lenalidomide is administered on days 1-21 of cycles 1-24 of the 28-day cycles at a dose of (or a dose of about) 25 mg.

In certain embodiments, the bispecific antibody, ibrutinib and/or lenalidomide are administered simultaneously. In some embodiments, lenalidomide, and the bispecific antibody are administered on the same day (e.g., on days 1, 8, and 15 of cycles 1-12) In some embodiments, ibrutinib, lenalidomide, and the bispecific antibody are administered on the same day (e.g., on days 1, 8, and 15 of cycles 1-21).

In some embodiments, the bispecific antibody, ibrutinib, and/or lenalidomide are administered sequentially.

In some embodiments ibrutinib (e.g., oral), lenalidomide (e.g., oral), and the bispecific antibody (e.g., subcutaneous) are administered in 28-day cycles, wherein:

(a) the bispecific antibody is administered as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2 and 3, a dose of 24 mg is administered on days 1, 8, 15, and 22;
(iii) in cycle 4 and onwards, a dose of 24 mg is administered on day 1;

(b) lenalidomide is administered on days 1-21 in cycle 1 and onwards, and

(c) ibrutinib is optionally administered on days 1-28 in cycle 1 and onwards.

In some embodiments lenalidomide (e.g., oral), and the bispecific antibody (e.g., subcutaneous) are administered in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-12, a dose of 24 mg is administered on day 1; and

(b) lenalidomide is administered orally at a dose of 25 mg/day on days 1-21 in cycles 1-12.

In some embodiments ibrutinib (e.g., oral), lenalidomide (e.g., oral), and the bispecific antibody (e.g., subcutaneous) are administered in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-24, a dose of 24 mg is administered on day 1;

(b) lenalidomide is administered orally at a dose of 20 mg/day on days 1-21 in cycles 1-24 and

(c) ibrutinib is administered orally at a dose of 560 mg/day on days 1-28 in cycles 1-24.

In some embodiments ibrutinib (e.g., oral), lenalidomide (e.g., oral), and the bispecific antibody (e.g., subcutaneous) are administered in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-24, a dose of 24 mg is administered on day 1;

(b) lenalidomide is administered orally at a dose of 20 mg/day on days 1-21 in cycles 1-24 and

(c) ibrutinib is administered orally at a dose of 420 mg/day on days 1-28 in cycles 1-24.

In some embodiments ibrutinib (e.g., oral), lenalidomide (e.g., oral), and the bispecific antibody (e.g., subcutaneous) are administered in 28-day cycles, wherein:

(a) the bispecific antibody is administered as follows:
(i) in cycle 1, a priming dose of 0.16 mg is administered on day 1, an intermediate dose of 0.8 mg is administered on day 8, and a dose of 48 mg is administered on days 15 and 22;
(ii) in cycles 2 and 3, a dose of 48 mg is administered on days 1, 8, 15, and 22;
(iii) in cycle 3 and onwards, a dose of 48 mg is administered on day 1;

(b) lenalidomide is administered on days 1-21 in cycles 1 and onwards; and

(c) ibrutinib is optionally administered on days 1-28 in cycle 1 and onwards.

In some embodiments lenalidomide (e.g., oral), and the bispecific antibody (e.g., subcutaneous) are administered in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycles 2 and 3, a dose of 48 mg is administered on days 1, 8, 15, and 22;
(iii) in cycles 4-12, a dose of 48 mg is administered on day 1; and

(b) lenalidomide is administered orally at a dose of 25 mg/day on days 1-21 in cycles 1-12.

In some embodiments ibrutinib (e.g., oral), lenalidomide (e.g., oral), and the bispecific antibody (e.g., subcutaneous) are administered in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycle 2 and 3, a dose of 48 mg is administered on days 1, 8, 15, and 22;
(iii) in cycles 4-24, a dose of 48 mg is administered on day 1;

(b) lenalidomide is administered orally at a dose of 20 mg/day on days 1-21 in cycles 1-24 and

(c) ibrutinib is administered orally at a dose of 560 mg/day on days 1-28 in cycles 1-24.

In some embodiments ibrutinib (e.g., oral), lenalidomide (e.g., oral), and the bispecific antibody (e.g., subcutaneous) are administered in 28-day cycles, wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycle 2 and 3, a dose of 48 mg is administered on days 1, 8, 15, and 22;
(iii) in cycles 4-24, a dose of 48 mg is administered on day 1;

(b) lenalidomide is administered orally at a dose of 20 mg/day on days 1-21 in cycles 1-24 and

(c) ibrutinib is administered orally at a dose of 420 mg/day on days 1-28 in cycles 1-24.

In one embodiment, dosing of the bispecific antibody and lenalidomide in 28-day cycles is as follows:

### Bispecific Antibody (Subcutaneous):

Cycle 1, day 1: Priming dose (0.16 mg)
Cycle 1, day 8: Intermediate dose (0.8 mg)
Cycle 1, days 15 and 22: Full dose (24 or 48 mg)
Cycles 2-3, days 1, 8, 15 and 22: Full dose (24 or 48 mg)
Cycles 4-12, day 1: Full dose (24 or 48 mg)
Lenalidomide (oral):
Cycles 1-12, days 1-21: 25 mg/day

In a further embodiment dosing of the bispecific antibody, ibrutinib and lenalidomide in 28-days cycles is as follows:

Bispecific antibody (subcutaneous):
Cycle 1, day 1: Priming dose (0.16 mg)
Cycle 1, day 8: Intermediate dose (0.8 mg)
Cycle 1, days 15 and 22: Full dose (24 or 48 mg)
Cycles 2-3, days 1, 8, 15 and 22: Full dose (24 or 48 mg)
Cycles 4-24, day 1: Full dose (24 or 48 mg)
Ibrutinib (oral):
Cycles 1-24, days 1-28: 420 mg/day or 560 mg/day
Lenalidomide (oral):
Cycles 1-24, days 1-21: 20 mg/day

In some embodiments, the subject has DLBCL is with histologically confirmed CD20+ disease.

In some embodiments, the DLBCL is high-grade B cell lymphoma with MYC and BCL-2 and/or BCL-6 translocations (double-hit or triple-hit).

In some embodiments, the DLBCL is follicular lymphoma Grade 3B.

In some embodiments, the DLBCL is relapsed and/or refractory DLBCL.

In some embodiments, the DLBCL has relapsed; i.e. has previously responded to prior therapy but has progressed after said prior therapy, progression having started 6 months or later, after completion of said prior therapy.

In some embodiments, the DLBCL is refractory; i.e. has either progressed during prior therapy, has failed to achieve an objective response to prior therapy, or has progressed within 6 months after completion of prior therapy, including maintenance therapy.

In some embodiments, the subject has relapsed or refractory disease to at least one prior systemic anti-lymphoma therapy, which contains an anti-CD20 monoclonal antibody.

In some embodiments, the DLBCL is not refractory to prior chimeric antigen receptor T cell (CAR-T) therapy.

In some embodiments, the subject has failed prior autologous stem cell transplant (ASCT) or is ineligible for ASCT.

In some embodiments, the subject is not refractory to lenalidomide or ibrutinib. In the context of this embodiment, refractoriness defined as:

Best response to prior regimen(s) of stable disease (SD) or progressive disease (PD), OR

Progressive disease within 6 months of completion of prior regimen(s)

In one embodiment, the subject has received at least 1 prior treatment with an anti-CD20 monoclonal antibody in combination with another systemic therapy.

In one embodiment, the subject has received prior CAR-T therapy or is ineligible for or unable to receive CAR-T therapy.

In a further embodiment, the subject has not had prior treatment with ibrutinib.

In some embodiments, the subject has an Eastern Cooperative Oncology Group (ECOG) performance status (ECOG PS) of 0, 1, or 2. Information regarding ECOG PS scores can be found in, e.g., Oken et al, Am J Clin Oncol 1982 December; 5(6):649-55).

In some embodiments, the subject has measurable disease as defined as (a)≥1 measurable nodal lesion (long axis >1.5 cm and short axis >1.0 cm) or ≥1 measurable extra-nodal lesion (long axis ≥1 cm) on CT or MRI.

In one embodiment, the subject has one or more measurable disease sites as defined as a positron emission tomography/computed tomography (PET/CT) scan demonstrating PET-positive lesion(s) and at least 1 measurable nodal lesion (long axis ≥1.5 cm and short axis >1.0 cm) or ≥1 measurable extra-nodal lesion (long axis ≥1.0 cm) on CT scan or MRI.

In some embodiments, the subject has laboratory values meeting the following criteria prior to receiving the first dose the first dose of the bispecific antibody:
Absolute neutrophil count (ANC)≥1.0×109/L (growth factor use is allowed if evidence of bone marrow involvement, but subject must not have received growth factor within 14 days prior to screening labs)
Hemoglobin ≥8.0 g/dL (RBC transfusions permitted, but subject must not have received blood transfusions within 7 days prior to screening labs)
Platelet count ≥75×109/L, or ≥50×109/L if bone marrow infiltration or splenomegaly (platelet transfusions permitted, but subject must not have received blood transfusions within 7 days prior to screening labs)
Serum aspartate transaminase (AST) or alanine transaminase (ALT) level ≤3×ULN
Total bilirubin level ≤1.5×ULN or ≤5×ULN for subjects with hepatic involvement of disease or of non-hepatic origin. Subjects with Gilbert's syndrome may have total bilirubin levels >1.5×ULN, but direct bilirubin must be <2×ULN
Estimated Creatinine Clearance (CrCl)≥50 mL/min (as calculated by Cockcroft-Gault Formula, modified as needed for factors such as body weight) Prothrombin time (PT)/International normalized ratio (INR)/Activated partial thromboplastin time (aPTT)≤1.5×ULN, unless receiving anticoagulation
In Further embodiments, the subject:
Must have diagnosis of DLBCL (de novo or histologically transformed from follicular lymphoma or nodal marginal zone lymphoma) with histologically confirmed CD20+ disease, inclusive of the following according to WHO 2016 classification and documented in pathology report:
Must have DLBCL, not otherwise specified (NOS)
Must have high-grade B cell lymphoma with MYC and BCL-2 and/or BCL-6 translocations per WHO 2016 (“double-hit” or “triple-hit”) Note: High-grade B-cell lymphomas NOS or other double-/triple-hit lymphomas (with histologies not consistent with DLBCL) are not eligible
Must have follicular lymphoma Grade 3B
Must have no prior treatment with a bispecific antibody targeting CD3 and CD20
Must have 1 or more measurable disease sites:
Must have a positron emission tomography/computed tomography (PET/CT) scan demonstrating PET-positive lesion(s) and at least 1 measurable nodal lesion (long axis ≥1.5 cm and short axis >1.0 cm) or ≥1 measurable extra-nodal lesion (long axis ≥1.0 cm) on CT scan or MRI
Must be eligible to receive and have a need for treatment initiation based on symptoms and/or disease burden as per investigator assessment.
must have Eastern Cooperative Oncology Group (ECOG) performance status 0-2.
Has no unresolved toxicities from prior anticancer therapy, defined as having not resolved to Common Terminology Criteria for Adverse Events (CTCAE, v 5.0), Grade 1, with the exception of alopecia.
Has no current evidence of primary central nervous system (CNS) tumor or known CNS involvement, including leptomeningeal disease, at screening.
Has no history of severe allergic or anaphylactic reactions to anti-CD20 mAb therapy or known significant allergy or intolerance to any component or excipient of epcoritamab or components of study drug combination agents (e.g., lenalidomide, ibrutinib, etc.)
Must not have had autologous stem cell transplantation within 3 months prior to screening.
Must not have had chemotherapy, non-investigational, or investigational anti-neoplastic agents (except CD20 mAbs) within 4 weeks or 5 half-lives (whichever is shorter) prior to the first dose of epcoritamab.
Has no clinically significant cardiovascular disease, including:
   Myocardial infarction or stroke within 6 months prior to enrollment,
   OR
   The following conditions within 3 months prior to enrollment: unstable or uncontrolled disease/condition related to or affecting cardiac function (e.g., unstable angina, congestive heart failure, New York Heart Association Class III-IV), uncontrolled cardiac arrhythmia
   OR
   Other clinically significant electrocardiogram (ECG) abnormalities within 6 months prior to enrollment unless deemed stable and appropriately treated.
Has no clinically significant liver disease, including hepatitis, current alcohol abuse, or cirrhosis.
Does not have active Hepatitis B Virus (HBV) or Hepatitis C Virus (HCV) infection.
Subjects who are positive for hepatitis B core antibody (HBcAb), hepatitis B surface antigen (HBsAg), or hepatitis C antibody must have a negative polymerase chain reaction (PCR) result before enrollment. Those who are PCR positive will be excluded.
Has no known history of Human Immunodeficiency Virus (HIV) infection. Note: HIV testing does not need to be conducted at screening unless it is required per local guidelines or institutional standards.
Has no known active bacterial, viral, fungal, mycobacterial, parasitic, or other infection (excluding fungal infections of the nail beds) requiring intravenous (IV) therapy or IV antibiotics within 2 weeks prior to enrollment.
Has no evidence of significant, uncontrolled concomitant diseases that could affect compliance with the protocol or interpretation of results.
Has no history of other prior malignancies, except for the following:
   Malignancy treated with curative intent and with no known active disease present for ≥3 years before the first dose of study drug and felt to be at low risk for recurrence by the treating physician
   Adequately treated non-melanoma skin cancer or lentigo maligna without evidence of disease
   Adequately treated carcinoma in situ without evidence of disease
   Localized prostate cancer, post-radical prostatectomy with non-rising prostate-specific antigen (PSA) levels <0.1 ng/mL Has not had radiation therapy to target lesion, or major surgery within 4 weeks of enrollment.
Has no Grade >1 neuropathy.
Must not have active tuberculosis (TB) or history of completed treatment for active TB within the past 12 months.
   Note: Interferon gamma release assay (IGRA) testing does not need to be performed at screening unless active or latent tuberculosis is suspected. For subjects with positive IGRA, active pulmonary tuberculosis must be excluded with clinical evaluation and radiologic imaging. Subjects with positive IGRA and no evidence of active disease may be enrolled after treatment for latent tuberculosis infection (recommendation isoniazid monotherapy for total of 6 months) has been initiated.
Has no evidence of CMV viremia (defined as any positive level above the lower limit of detection) at screening.
Has no current autoimmune disease requiring immunosuppressive therapy except for up to 20 mg prednisone daily (or equivalent).
Has no life-threatening illness, medical condition, or organ system dysfunction that, in the Investigator's opinion, could compromise the subject's safety or put the study outcomes at undue risk.
Has no current seizure disorder requiring therapy.
Has no known active SARS-CoV-2 infection. If a subject has signs/symptoms suggestive of SARS-CoV-2 infection or have recent known exposure to someone with SARS-CoV infection, they should undergo molecular (e.g., PCR) testing or 2 negative antigen test results at least 24 hours apart to rule out SARS-CoV-2 infection.
Subjects who do not meet SARS-CoV-2 infection eligibility criteria must be screen failed and may only rescreen after they meet the following SARS-CoV-2 infection viral clearance criteria:
   At least 10 days since first positive test result have passed in asymptomatic patients or at least 10 days since recovery, defined as resolution of fever without use of antipyretics and improvement in symptoms.
Must not have had major surgery within 4 weeks of the first dose of study drug.

In one embodiment, the subject has no current evidence of primary central nervous system (CNS) tumor or known CNS involvement, including leptomeningeal disease, at screening

The subject may have no history of severe allergic or anaphylactic reactions to anti-CD20 monoclonal antibody therapy or known significant allergy or intolerance to any component or excipient of epcoritamab or components of study drug combination agents (e.g., lenalidomide, ibrutinib, etc.)

In one embodiment, the subject must not have had autologous stem cell transplantation within 3 months prior to screening.

In one embodiment, the subject must not have had chemotherapy, non-investigational, or investigational anti-neoplastic agents (except CD20 monoclonal antibodies) within 4 weeks or 5 half-lives (whichever is shorter) prior to the first dose of epcoritamab.

In one embodiment, the subject has no clinically significant cardiovascular disease, including:
Myocardial infarction or stroke within 6 months prior to enrollment,
OR
The following conditions within 3 months prior to enrollment: unstable or uncontrolled disease/condition related to or affecting cardiac function (e.g., unstable angina, congestive heart failure, New York Heart Association Class III-IV), uncontrolled cardiac arrhythmia
OR
Other clinically significant electrocardiogram (ECG) abnormalities within 6 months prior to enrollment unless deemed stable and appropriately treated.
Left ventricular ejection fraction (LVEF) must be within institutional normal limits by multi-gated acquisition (MUGA) or transthoracic echocardiography at screening.

In one embodiment, the subject has no history of other prior malignancies, except for the following:
Malignancy treated with curative intent and with no known active disease present for ≥3 years before the first dose of study drug and felt to be at low risk for recurrence by treating physician
Adequately treated non-melanoma skin cancer or lentigo maligna without evidence of disease
Adequately treated carcinoma in situ without evidence of disease;
Localized prostate cancer, post-radical prostatectomy with non-rising prostate-specific antigen (PSA) levels <0.1 ng/mL

In one embodiment, the subject has not had radiation therapy to target lesion, or major surgery within 4 weeks of enrollment.

In one embodiment, the subject has no Grade >1 neuropathy

A human subject receiving a treatment described herein may be a patient having one or more of the inclusion criteria set forth in Example 3, or not having one or more of the exclusion criteria set forth in Example 3.

The methods described herein are advantageous for treating Diffuse large B-cell lymphoma, such as relapsed and/or refractory Diffuse large B-cell lymphoma. The treatment is maintained continuously using, e.g., the treatment regimens described herein. However, treatment may be terminated when progressive disease develops or unacceptable toxicity occurs.

The response of subjects with Diffuse large B-cell lymphoma to treatment using the methods described herein may be assessed according to the Lugano Response Criteria for Malignant Lymphoma (also referred to as “Lugano criteria” herein) and/or Lymphoma Response to Immunomodulatory Therapy Criteria (also referred to as “LYRIC” herein), as described in Example 3. In one embodiment, complete response (CR), partial response (PR), and stable disease (SD) are assessed using the Lugano criteria. In some embodiments, patients showing disease progression, also referred to as progressive disease (PD), according to the Lugano criteria are further evaluated according to LYRIC. Details regarding the Lugano criteria/classification system, including definitions for complete response, partial response, no response/stable disease, and progressive disease are provided in Cheson et al. J Clin Oncol 2014; 32:3059-68 (see, in particular, Table 3 in Cheson et al., 2014). Details regarding Lugano are provided in Example 2 herein.

In some embodiments, subjects are treated with the methods described herein until they show disease progression (PD), e.g., as defined by Lugano criteria and/or LYRIC. In one embodiment, subjects are treated with the methods described herein until they show disease progression (PD) as defined by both Lugano criteria and LYRIC.

Subjects treated according to the methods described herein preferably experience improvement in at least one sign of Diffuse large B-cell lymphoma. In one embodiment, improvement is measured by a reduction in the quantity and/or size of measurable tumor lesions. In some embodiments, lesions can be measured on CT (computed tomography), PET-CT (positron emission tomography-computed tomography), or MRI (magnetic resonance imaging) films. In some embodiments, cytology or histology can be used to evaluate responsiveness to a therapy. In some embodiments, bone marrow aspirate, bone marrow biopsy, tumor biopsy, physical examination and/or laboratory tests (e.g., tumor cells in ascites or pleural fluid) can be used to evaluate response to therapy.

In one embodiment, the subject treated exhibits a complete response (CR), a partial response (PR), or stable disease (SD), as defined by the Lugano criteria or LYRIC (see, e.g., Example 2 herein). In some embodiments, the methods described herein produce at least one therapeutic effect chosen from prolonged survival, such as progression-free survival or overall survival, optionally compared to another therapy, such as treatment with lenalidomide, or lenalidomide and ibrutinib, alone.

In one embodiment, the bispecific antibody used in the methods described herein is administered subcutaneously, and thus is formulated in a pharmaceutical composition such that it is compatible with subcutaneous (s.c.) administration, i.e., having a formulation and/or concentration that allows pharmaceutical acceptable s.c. administration at the doses described herein. In some embodiments, subcutaneous administration is carried out by injection. For example, formulations for Duobody® CD3×CD20 that are compatible with subcutaneous formulation and can be used in the methods described herein have been described previously (see, e.g., WO2019155008, which is incorporated herein by reference). In some embodiments, the bispecific antibody may be formulated using sodium acetate trihydrate, acetic acid, sodium hydroxide, sorbitol, polysorbate 80, and water for injection, and have a pH of 5.5 or about 5.5.

In some embodiments, the bispecific antibody is provided as a 5 mg/mL or 60 mg/mL concentrate. In other embodiments, the desired dose of the bispecific antibody is reconstituted to a volume of about 1 mL for subcutaneous injection.

In one embodiment, a suitable pharmaceutical composition for the bispecific antibody can comprise the bispecific antibody, 20-40 mM acetate, 140-160 mM sorbitol, and a surfactant, such as polysorbate 80, and having a pH of 5.3-5.6. In another embodiment, the pharmaceutical formulation may comprise an antibody concentration in the range of 5-100 mg/mL, e.g., 48 or 60 mg/mL of the bispecific antibody, 30 mM acetate, 150 mM sorbitol, 0.04% w/v polysorbate 80, and have a pH of 5.5. Such a formulation may be diluted with, e.g., the formulation buffer to allow proper dosing and subcutaneous administration.

The volume of the pharmaceutical composition is appropriately selected to allow for subcutaneous administration of the antibody. For example, the volume to be administered is in the range of about 0.3 mL to about 3 mL, such as from 0.3 mL to 3 mL. The volume to be administered can be 0.5 mL, 0.8 mL, 1 mL, 1.2 mL, 1.5 ml, 1.7 mL, 2 mL, or 2.5 mL, or about 0.5 mL, about 0.8 mL, about 1 mL, about 1.2 mL, about 1.5 ml, about 1.7 mL, about 2 mL, or about 2.5 mL. Accordingly, in one embodiment, the volume to be administered is 0.5 mL or about 0.5 mL. In some embodiments, the volume to be administered is 0.8 mL or about 0.8 mL.

In some embodiments, the volume to be administered is 1 mL or about 1 mL. In some embodiments, the volume to be administered is 1.2 mL or about 1.2 mL. In some embodiments, the volume to be administered is 1.5 mL or about 1.5 mL. In some embodiments, the volume to be administered is 1.7 mL or about 1.7 mL. In some embodiments, the volume to be administered is 2 mL or about 2 mL. In some embodiments, the volume to be administered is 2.5 mL or about 2.5 mL.

In one embodiment, ibrutinib is formulated in a pharmaceutical composition comprising pharmaceutically-acceptable excipients for administration (e.g., oral administration) in accordance with local standard-of-care practice, e.g., as specified by local guidelines or local product labels. For example, in some embodiments, ibrutinib is provided in an oral dosage form, e.g., a capsule.

In one embodiment, lenalidomide is formulated in a pharmaceutical composition comprising pharmaceutically-acceptable excipients suitable for administration (e.g., oral administration), e.g., in accordance with local standard-of-care practice, e.g., as specified by local guidelines or local product labels. In some embodiments, lenalidomide is formulated in an oral dosage form, e.g., a capsule. In some embodiments, lenalidomide is formulated as a capsule comprising lenalidomide, lactose anhydrous, microcrystalline cellulose, croscarmellose sodium, and magnesium stearate.

In one embodiment, the bispecific antibody used in the methods described herein comprises:

(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences within the amino acid sequence of SEQ ID NO: 6, and the VL region comprises the CDR1, CDR2 and CDR3 sequences within the amino acid sequence of SEQ ID NO: 7; and

(ii) a second binding arm comprising a second antigen-binding region which binds to human CD20 and comprises a VH region and a VL region, wherein the VH region comprises the CDR1, CDR2 and CDR3 sequences within the amino acid sequence of SEQ ID NO: 13, and the VL region comprises the CDR1, CDR2 and CDR3 sequences within the amino acid sequence SEQ ID NO: 14.

CDR1, CDR2 and CDR3 regions can be identified from variable heavy and light chain regions using methods known in the art. The CDR regions from said variable heavy and light chain regions can be annotated according to IMGT (see Lefranc et al., Nucleic Acids Research 1999; 27:209-12, 1999] and Brochet. Nucl Acids Res 2008; 36:W503-8).

In some embodiments, the bispecific antibody comprises:

(i) a first binding arm comprising a first antigen-binding region which binds to human CD3ε (epsilon) and comprises VHCDR1, VHCDR2 and VHCDR3 the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and

(ii) a second binding arm comprising a second antigen-binding region which binds to human CD20 and comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.

In some embodiments, the bispecific antibody comprises:

(i) a first binding arm comprising a first antigen-binding region which binds to human CD3ε (epsilon) and comprises a VH region comprising the amino acid sequence of SEQ ID NO: 6, and a VL region comprising the amino acid sequence of SEQ ID NO: 7; and

(ii) a second binding arm comprising a second antigen-binding region which binds to human CD20 and comprises a VH region comprising the amino acid sequence of SEQ ID NO: 13, and a VL region comprising the amino acid sequence of SEQ ID NO: 14.

In one embodiment, the bispecific antibody is a full-length antibody. In some embodiments, the bispecific antibody have an inert Fc region. In some embodiments, the bispecific antibody is a full-length antibody and have an inert Fc region. In some embodiments, the first binding arm for CD3 is derived from a humanized antibody, e.g., from a full-length IgG1,λ (lambda) antibody such as H1L1 described in WO2015001085, which is incorporated herein by reference, and/or the second binding arm for CD20 is derived from a human antibody, e.g., from a full-length IgG1,κ (kappa) antibody such as clone 7D8 as described in WO2004035607, which is incorporated herein by reference. The bispecific antibody may be produced from two half molecule antibodies, wherein each of the two half molecule antibodies comprising, e.g., the respective first and second binding arms set forth in SEQ ID NOs: 24 and 25, and SEQ ID NOs: 26 and 27. The half-antibodies may be produced in CHO cells and the bispecific antibodies generated by, e.g., Fab-arm exchange. In one embodiment, the bispecific antibody is a functional variant of DuoBody® CD3×CD20.

Accordingly, in some embodiments, the bispecific antibody comprises (i) a first binding arm comprising a first antigen-binding region which binds to human CD3ε (epsilon) and comprises a VH region comprising an amino acid sequence which is at least 85%, 90%, 95%, 96%, 97%, 98%, or 99% identical to SEQ ID NO: 6 or a VH region comprising the amino acid sequence of SEQ ID NO: 6, but with 1, 2, or 3 mutations (e.g., amino acid substitutions), and a VL region comprising an amino acid sequence which is at least 85%, 90%, 95%, 96%, 97%, 98%, or 99% identical to SEQ ID NO: 7 or a VL region comprising the amino acid sequence of SEQ ID NO: 7, but with 1, 2, or 3 mutations (e.g., amino acid substitutions); and (ii) a second binding arm comprising a second antigen-binding region which binds to human CD20 and comprises a VH region comprising an amino acid sequence which is at least 85%, 90%, 95%, 98%, or 99% identical to SEQ ID NO: 13 or a VH region comprising the amino acid sequence of SEQ ID NO: 13, but with 1, 2, or 3 mutations (e.g., amino acid substitutions), and a VL region comprising an amino acid sequence which is at least 85%, 90%, 95%, 98%, or 99% identical to SEQ ID NO: 14 or a VL region comprising the amino acid sequence of SEQ ID NO: 14, but with 1, 2, or 3 mutations (e.g., amino acid substitutions).

In one embodiment, the bispecific antibody comprises:

(i) a first binding arm comprising a first antigen-binding region which binds to human CD3ε (epsilon) and comprises a heavy chain comprising the amino acid sequence of SEQ ID NO: 24, and a light chain comprising the amino acid sequence of SEQ ID NO: 25; and

(ii) a second binding arm comprising a second antigen-binding region which binds to human CD20 and comprises a VH region comprising the amino acid sequence of SEQ ID NO: 26, and a VL region comprising the amino acid sequence of SEQ ID NO: 27.

In some embodiments, the bispecific antibody comprises (i) a first binding arm comprising a first antigen-binding region which binds to human CD3ε (epsilon) and comprises a heavy chain comprising an amino acid sequence which is at least 85%, 90%, 95%, 98%, or 99% identical to SEQ ID NO: 24 or a heavy chain comprising the amino acid sequence of SEQ ID NO: 24, but with 1, 2, or 3 mutations (e.g., amino acid substitutions), and a light chain comprising an amino acid sequence which is at least 85%, 90%, 95%, 98%, or 99% identical to SEQ ID NO: 25 or a light chain region comprising the amino acid sequence of SEQ ID NO: 25, but with 1, 2, or 3 mutations (e.g., amino acid substitutions); and

(ii) a second binding arm comprising a second antigen-binding region which binds to human CD20 and comprises a heavy chain comprising an amino acid sequence which is at least 85%, 90%, 95%, 98%, or 99% identical to SEQ ID NO: 26 or a heavy chain comprising the amino acid sequence of SEQ ID NO: 26, but with 1, 2, or 3 mutations (e.g., amino acid substitutions), and a light chain comprising an amino acid sequence which is at least 85%, 90%, 95%, 98%, or 99% identical to SEQ ID NO: 27 or a light chain region comprising the amino acid sequence of SEQ ID NO: 27, but with 1, 2, or 3 mutations (e.g., amino acid substitutions).

Various constant regions or variants thereof may be used in the bispecific antibody. In one embodiment, the antibody comprises an IgG constant region, such as a human IgG1 constant region, e.g., a human IgG1 constant region as defined in SEQ ID NO: 15, or any other suitable IgG1 allotype. In some embodiments, the bispecific antibody is a full-length antibody with a human IgG1 constant region. In some embodiments, the first binding arm of the bispecific antibody is derived from a humanized antibody, preferably from a full-length IgG1,λ (lambda) antibody. In one embodiment, the first binding arm of the bispecific antibody is derived from a humanized antibody, e.g., from a full-length IgG1) (lambda) antibody, and thus comprises a λ light chain constant region. In some embodiments, the first binding arm comprises a λ light chain constant region as defined in SEQ ID NO: 22. In some embodiments, the second binding arm of the bispecific antibody is derived from a human antibody, preferably from a full-length IgG1,κ (kappa) antibody. In some embodiments, the second binding arm of the bispecific antibody is derived from a human antibody, preferably from a full-length IgG1,κ (kappa) antibody, and thus may comprise a κ light chain constant region. In some embodiments, the second binding arm comprises a κ light chain constant region as defined in SEQ ID NO: 23. In a preferred embodiment, the first binding arm comprises a λ light chain constant region as defined in SEQ ID NO: 22 and the second binding arm comprises a κ light chain constant region as defined in SEQ ID NO: 23.

It is understood that the constant region portion of the bispecific antibody may comprise modifications that allow for efficient formation/production of bispecific antibodies and/or provide for an inert Fc region. Such modifications are well known in the art.

Different formats of bispecific antibodies are known in the art (reviewed by Kontermann, Drug Discov Today 2015; 20:838-47; MAbs, 2012; 4:182-97). Thus, the bispecific antibody used in the methods and uses described herein are not limited to any particular bispecific format or method of producing it. For example, bispecific antibodies may include, but are not limited to, bispecific antibodies with complementary CH3 domains to force heterodimerization, Knobs-into-Holes molecules (Genentech, WO9850431), CrossMAbs (Roche, WO2011117329), or electrostatically-matched molecules (Amgen, EP1870459 and WO2009089004; Chugai, US201000155133; Oncomed, WO2010129304).

Preferably, the bispecific antibody comprises an Fc-region comprising a first heavy chain with a first Fc sequence comprising a first CH3 region, and a second heavy chain with a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions. Further details on these interactions and how they can be achieved are provided in e.g. WO2011131746 and WO2013060867 (Genmab), which are hereby incorporated by reference. In one embodiment, the bispecific antibody comprises in the first heavy chain (i) the amino acid L in the position corresponding to F405 in the human IgG1 heavy chain constant region of SEQ ID NO: 15, and comprises in the second heavy chain the amino acid R in the position corresponding to K409 in the human IgG1 heavy chain constant region of SEQ ID NO: 15, or vice versa.

Bispecific antibodies may comprise modifications in the Fc region to render the Fc region inert, or non-activating. Thus, in the bispecific antibodies disclosed herein, one or both heavy chains may be modified so that the antibody induces Fc-mediated effector function to a lesser extent relative to the bispecific antibody which does not have the modification. Fc-mediated effector function may be measured by determining Fc-mediated CD69 expression on T cells (i.e. CD69 expression as a result of CD3 antibody-mediated, Fcγ receptor-dependent CD3 crosslinking), by binding to Fey receptors, by binding to C1q, or by induction of Fc-mediated cross-linking of FcγRs. In particular, the heavy chain constant region sequence may be modified so that Fc-mediated CD69 expression is reduced by at least 50%, at least 60%, at least 70%, at least 80%, at least 90%, at least 99% or 100% when compared to a wild-type (unmodified) antibody, wherein said Fc-mediated CD69 expression is determined in a PBMC-based functional assay, e.g. as described in Example 3 of WO2015001085. Modifications of the heavy and light chain constant region sequences may also result in reduced binding of C1q to said antibody. As compared to an unmodified antibody, the reduction may be by at least 70%, at least 80%, at least 90%, at least 95%, at least 97%, or 100%, and C1q binding may be determined, e.g., by ELISA. Further, the Fc region which may be modified so that the antibody mediates reduced Fc-mediated T-cell proliferation compared to an unmodified antibody by at least 50%, at least 60%, at least 70%, at least 80%, at least 90%, at least 99% or 100%, wherein said T-cell proliferation is measured in a PBMC-based functional assay. Examples of amino acid positions that may be modified, e.g., in an IgG1 isotype antibody, include positions L234 and L235. Thus, in one embodiment, the bispecific antibody may comprise a first heavy chain and a second heavy chain, and wherein in both the first heavy chain and the second heavy chain, the amino acid residues at the positions corresponding to positions L234 and L235 in a human IgG1 heavy chain according to Eu numbering are F and E, respectively. In addition, a D265A amino acid substitution can decrease binding to all Fcγ receptors and prevent ADCC (Shields et al., JBC 2001; 276:6591-604). Therefore, the bispecific antibody may comprise a first heavy chain and a second heavy chain, wherein in both the first heavy chain and the second heavy chain, the amino acid residue at the position corresponding to position D265 in a human IgG1 heavy chain according to Eu numbering is A.

In one embodiment, in the first heavy chain and second heavy chain of the bispecific antibody, the amino acids in the positions corresponding to positions L234, L235, and D265 in a human IgG1 heavy chain, are F, E, and A, respectively. An antibody having these amino acids at these positions is an example of an antibody having an inert Fc region, or a non-activating Fc region.

In some embodiments, the bispecific antibody comprises a first heavy chain and a second heavy chain, wherein in both the first and second heavy chains, the amino acids in the positions corresponding to positions L234, L235, and D265 in the human IgG1 heavy chain constant region of SEQ ID NO: 15 are F, E, and A, respectively. In some embodiments, the bispecific antibody comprises a first heavy chain and a second heavy chain, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa. In a preferred embodiment, the bispecific antibody comprises a first heavy chain and a second heavy chain, wherein (i) in both the first and second heavy chains, the amino acids in the positions corresponding to positions L234, L235, and D265 in the human IgG1 heavy chain constant region of SEQ ID NO: 15 are F, E, and A, respectively, and (ii) in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.

With regards to the bispecific antibodies described herein, those which have the combination of three amino acid substitutions L234F, L235E and D265A and in addition the K409R or the F405L mutation, as described above, may be referred to with the suffix “FEAR” or “FEAL”, respectively.

An amino acid sequence of a wild type IgG1 heavy chain constant region may be identified herein as SEQ ID NO: 15. Consistent with the embodiments disclosed above, the bispecific antibody may comprise an IgG1 heavy chain constant region carrying the F405L substitution and may have the amino acid sequence set forth in SEQ ID NO: 17 and/or an IgG1 heavy chain constant region carrying the K409R substitution and may have the amino acid sequence set forth in SEQ ID NO: 18, and have further substitutions that render the Fc region inert or non-activating. Hence, in one embodiment, the bispecific antibody comprises a combination of IgG1 heavy chain constant regions, with the amino acid sequence of one of the IgG1 heavy chain constant regions carrying the L234F, L235E, D265A and F405L substitutions (e.g., as set forth in SEQ ID NO: 19) and the amino acid sequence of the other IgG1 heavy chain constant region carrying the L234F, L235E, D265A and K409R substitutions (e.g., as set forth in SEQ ID NO: 20). Thus, in some embodiments, the bispecific antibody comprises heavy chain constant regions comprising the amino acid sequences of SEQ ID NOs: 19 and 20.

In preferred embodiments, the bispecific antibody used in the methods and uses described herein comprises a first binding arm comprising a heavy chain and a light chain as defined in SEQ ID NOs: 24 and 25, respectively, and a second binding arm comprising a heavy chain and a light chain as defined in SEQ ID NOs: 26 and 27, respectively. Such an antibody is referred to herein as DuoBody® CD3×CD20. Also, variants of such antibodies are contemplated use in the methods and uses as described herein. In some embodiment, the bispecific antibody comprising a heavy chain and a light chain consisting of the amino acid sequences set forth in SEQ ID NOs: 24 and 25, respectively, and a heavy chain and a light chain consisting of the amino acid sequences set forth in SEQ ID NOs: 26 and 27, respectively. In some embodiments, the bispecific antibody is epcoritamab (CAS 2134641-34-0), or a biosimilar thereof.

### Medical Use

Further provided herein is a bispecific antibody for use in a method as disclosed above.

In particular embodiments, the bispecific antibody is for use in a method of treating diffuse large B-cell lymphoma (DLBCL) in a human subject, wherein the bispecific antibody is administered to a subject in combination with an effective amount of lenalidomide and optionally, an effective amount of ibrutinib, wherein the bispecific antibody comprises:

(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and

(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;

wherein the bispecific antibody is administered at a dose of 24 mg or 48 mg, and wherein lenalidomide, the bispecific antibody and optionally ibrutinib are administered in 28-day cycles.

Also provided herein is a bispecific antibody for the manufacture of a medicament for use in a method as disclosed above.

In particular, the bispecific antibody is for the manufacture of a medicament for use in treating diffuse large B-cell lymphoma (DLBCL) in a human subject, wherein the bispecific antibody is administered to the subject in combination with an effective amount of lenalidomide and optionally, an effective amount of ibrutinib, wherein the bispecific antibody comprises:

(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and

(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;

wherein the bispecific antibody is administered at a dose of 24 mg or 48 mg, and wherein lenalidomide, the bispecific antibody and optionally ibrutinib are administered in 28-day cycles.

### Kits

Also provided herein are kits which include a pharmaceutical composition containing a bispecific antibody which binds to CD3 and CD20 in accordance with the invention, such as DuoBody® CD3×CD20 or epcoritamab, and a pharmaceutically acceptable carrier, in a therapeutically effective amount adapted for use in the methods described herein. The kits may also include a pharmaceutical composition containing ibrutinib (e.g., for oral administration) and/or lenalidomide (e.g., for oral administration). The kits may further include a pharmaceutical composition containing lenalidomide (e.g. for oral administration). The kits optionally also can include instructions, e.g., comprising administration schedules, to allow a practitioner (e.g., a physician, nurse, or patient) to administer the composition or compositions contained therein to a patient with Diffuse Large B-Cell Lymphoma. The kit also can include a syringe or syringes.

Optionally, the kits include multiple packages of the single-dose pharmaceutical compositions each containing an effective amount of the bispecific antibody for a single administration in accordance with the methods described herein. They may also include multiple packages of single dose pharmaceutical compositions containing a dose of ibrutinib and/or lenalidomide in accordance with a standard of practice regimen. Instruments or devices necessary for administering the pharmaceutical composition(s) also may be included in the kits.

### Further Embodiments

1. A method of treating Diffuse large B-cell lymphoma (DLBCL) in a human subject, the method comprising administering to the subject a bispecific antibody and an effective amount of lenalidomide and optionally, an effective amount of ibrutinib, wherein the bispecific antibody comprises:

(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and

(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;

wherein the bispecific antibody is administered at a dose of 24 mg or 48 mg, and wherein, lenalidomide, the bispecific antibody and optionally ibrutinib are administered in 28-day cycles.

2. The method of embodiment 1, wherein the bispecific antibody is administered at a dose of 24 mg.
3. The method of embodiment 1, wherein the bispecific antibody is administered at a dose of 48 mg.
4. The method of any one of embodiments 1-3, wherein the bispecific antibody is administered once every week (weekly administration).
5. The method of embodiment 4, wherein the weekly administration of 24 mg or 48 mg is performed for 2.5 28-day cycles.
6. The method of embodiment 4 or 5, wherein after the weekly administration, the bispecific antibody is administered once every four weeks, such as in 28-day cycles, on day 1 of each 28-day cycle.
7. The method of embodiment 6, wherein the administration once every four weeks is performed for at least eight 28-day cycles, such as eight 28-day cycles.
8. The method of embodiment 6, wherein the administration once every four weeks is performed for at least twenty 28-day cycles, such as twenty 28-day cycles.
9. The method of any one of embodiments 4-8, wherein prior to the weekly administration of 24 mg or 48 mg, a priming dose of the bispecific antibody is administered in cycle 1 of the 28-day cycles.
10. The method of embodiment 9, wherein the priming dose is administered two weeks prior to administering the first weekly dose of 24 mg or 48 mg.
11. The method of embodiment 9 or 10, wherein the priming dose is 0.16 mg.
12. The method of any one of embodiments 9-11, wherein after administering the priming dose and prior to administering the first weekly dose of 24 mg or 48 mg, an intermediate dose of the bispecific antibody is administered.
13. The method of embodiment 12, wherein the priming dose is administered on day 1 and the intermediate dose is administered on day 8 before the first weekly dose of 24 mg or 48 mg on days 15 and 22 of cycle 1.
14. The method of embodiment 12 or 13, wherein the intermediate dose is 0.8 mg.
15. The method of any one of embodiments 1-14, wherein lenalidomide is administered once a day from day 1 to day 21 of the 28-day cycles.
16. The method of any one of embodiments 1-15, wherein lenalidomide is administered from cycle 1 to cycle 12 of the 28-day cycles.
17. The method of any one of embodiments 1-15, wherein lenalidomide is administered from cycle 1 to cycle 24 of the 28-day cycles.
18. The method of any one of embodiments 1-17, wherein lenalidomide is administered at a dose of 20 to 30 mg, such as 25 mg.
19. The method of any one of embodiments 1-17, wherein lenalidomide is administered at a dose of 20 to 30 mg in cycle 1 to cycle 12 of the 28-day cycles.
20. The method of any one of embodiments 1-17, wherein lenalidomide is administered at a dose of 25 mg in cycle 1 to cycle 12 of the 28-day cycles.
21. The method of any one of embodiments 1-14, wherein lenalidomide is administered at a dose of 10 to 25 mg, such as 25 mg.
22. The method of any one of embodiments 1-14 and 21, wherein lenalidomide is administered at a dose of 10 to 25 mg in cycle 1 to cycle 24 of the 28-day cycles.
23. The method of any one of embodiments 1-14 and 21-22, wherein lenalidomide is administered at a dose of 20 mg in cycle 1 to cycle 24 of the 28-day cycles.
24. The method of any one of embodiments 1-14 and 21-23, wherein ibrutinib is administered once a day from day 1 to day 28 of the 28 day cycles.
25. The method of any one of embodiments 1-14 and 21-24, wherein ibrutinib is administered from cycle 1 to cycle 24 of the 28 days cycles.
26. The method of any one of embodiments 1-14 and 21-25, wherein ibrutinib is administered at a dose of 280 to 560 mg, such as 280, 420 or 560 mg.
27. The method of any one of embodiments 1-14 and 21-25, wherein ibrutinib is administered at a dose of 560 mg in cycle 1 to cycle 24 of the 28 days cycles, or at a dose of 420 mg in cycle 1 to cycle 24 of the 28 days cycles.
28. The method of any one of embodiments 1, 2, and 4-27, wherein administration is performed in 28-day cycles, and wherein:

(a) the bispecific antibody is administered as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2 and 3, a dose of 24 mg is administered on days 1, 8, 15, and 22;
(iii) in cycle 4 and onwards, a dose of 24 mg is administered on day 1;

(b) lenalidomide is administered on days 1-21 in cycle 1 and onwards, and

(c) ibrutinib is optionally administered on days 1-28 in cycle 1 and onwards.

29. The method of any one of embodiments 1, 2, and 4-28, wherein administration is performed in 28-day cycles, and wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-12, a dose of 24 mg is administered on day 1; and

(b) lenalidomide is administered orally at a dose of 25 mg/day on days 1-21 in cycles 1-12.

30. The method of any one of embodiments 1, 2, and 4-29, wherein administration is performed in 28-day cycles, and wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-24, a dose of 24 mg is administered on day 1;

(b) lenalidomide is administered orally at a dose of 20 mg/day on days 1-21 in cycles 1-24 and

(c) ibrutinib is administered orally at a dose of 560 mg/day on days 1-28 in cycles 1-24.

31. The method of any one of embodiments 1, 2, and 4-29, wherein administration is performed in 28-day cycles, and wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-24, a dose of 24 mg is administered on day 1;

(b) lenalidomide is administered orally at a dose of 20 mg/day on days 1-21 in cycles 1-24 and

(c) ibrutinib is administered orally at a dose of 420 mg/day on days 1-28 in cycles 1-24.

32. The method of any one of embodiments 1 and 3-27, wherein administration is performed in 28-day cycles, and wherein:

(a) the bispecific antibody is administered as follows:
(i) in cycle 1, a priming dose of 0.16 mg is administered on day 1, an intermediate dose of 0.8 mg is administered on day 8, and a dose of 48 mg is administered on days 15 and 22;
(ii) in cycles 2 and 3, a dose of 48 mg is administered on days 1, 8, 15, and 22;
(iii) in cycle 3 and onwards, a dose of 48 mg is administered on day 1;

(b) lenalidomide is administered on days 1-21 in cycles 1 and onwards; and

(c) ibrutinib is optionally administered on days 1-28 in cycle 1 and onwards.

33. The method of any one of embodiments 1, 3-27 and 31, wherein administration is performed in 28-day cycles, and wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycles 2 and 3, a dose of 48 mg is administered on days 1, 8, 15, and 22;
(iii) in cycles 4-12, a dose of 48 mg is administered on day 1; and

(b) lenalidomide is administered orally at a dose of 25 mg/day on days 1-21 in cycles 1-12.

34. The method of any one of embodiments 1, 3-27 and 31-33, wherein administration is performed in 28-day cycles, and wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycle 2 and 3, a dose of 48 mg is administered on days 1, 8, 15, and 22;
(iii) in cycles 4-24, a dose of 48 mg is administered on day 1;

(b) lenalidomide is administered orally at a dose of 20 mg/day on days 1-21 in cycles 1-24 and

(c) ibrutinib is administered orally at a dose of 560 mg/day on days 1-28 in cycles 1-24.

35. The method of any one of embodiments 1, 3-27 and 31-32, wherein administration is performed in 28-day cycles, and wherein:

(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycle 2 and 3, a dose of 48 mg is administered on days 1, 8, 15, and 22;
(iii) in cycles 4-24, a dose of 48 mg is administered on day 1;

(b) lenalidomide is administered orally at a dose of 20 mg/day on days 1-21 in cycles 1-24 and

(c) ibrutinib is administered orally at a dose of 420 mg/day on days 1-28 in cycles 1-24.

36. The method of any one of embodiments 1-35, wherein the bispecific antibody is administered subcutaneously.
37. The method of any one of embodiments 1-36, wherein ibrutinib is administered orally.
38. The method of any one of embodiments 1-37, wherein lenalidomide is administered orally.
39. The method of any one of embodiments 1-38, wherein the bispecific antibody, lenalidomide and optionally ibrutinib are administered sequentially.
40. The method of any one of embodiments 1-39, wherein the DLBCL is with histologically confirmed CD20+ disease.
41. The method of any one of embodiments 1-40, wherein the DLBCL is high-grade B cell lymphoma with MYC and BCL-2 and/or BCL-6 translocations (double-hit or triple-hit).
42. The method of any one of embodiments 1-41, wherein the DLBCL is follicular lymphoma Grade 3B.
43. The method of any one of embodiments 1-42, wherein the DLBCL is relapsed and/or refractory DLBCL.
44. The method of any one of embodiments 1-43, wherein the DLBCL has relapsed; i.e. has previously responded to prior therapy but has progressed after said prior therapy, progression having started 6 months or later, after completion of said prior therapy.
45. The method of any one of embodiments 1-44, wherein the DLBCL is refractory; i.e. has either progressed during prior therapy, has failed to achieve an objective response to prior therapy, or has progressed within 6 months after completion of prior therapy, including maintenance therapy.
46. The method of any one of embodiments 1-45, wherein the subject has relapsed or refractory disease to at least one prior systemic anti-lymphoma therapy, which contains an anti-CD20 monoclonal antibody.
47. The method of any one of embodiments 1-46, wherein the DLBCL is not refractory to prior chimeric antigen receptor T cell (CAR-T) therapy.
48. The method of any one of embodiments 1-46, wherein the subject is not refractory to lenalidomide or ibrutinib.
49. The method of any one of embodiments 1-48, wherein the subject has received at least 1 prior treatment with an anti-CD20 monoclonal antibody in combination with another systemic therapy.
50. The method of any one of embodiments 1-49, wherein the subject has received prior CAR-T therapy or is ineligible for or unable to receive CAR-T therapy.
51. The method of any one of embodiments 1-50, wherein the subject has not had prior treatment with ibrutinib.
52. The method of any one of embodiments 1-51, wherein:
(i) the first antigen-binding region of the bispecific antibody comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and
(ii) the second antigen-binding region of the bispecific antibody comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.
   53. The method of any one of embodiments 1-52, wherein:
(i) the first antigen-binding region of the bispecific antibody comprises a VH region comprising the amino acid sequence of SEQ ID NO: 6, and the VL region comprising the amino acid sequence of SEQ ID NO: 7; and
(ii) the second antigen-binding region of the bispecific antibody comprises a VH region comprising the amino acid sequence of SEQ ID NO: 13, and the VL region comprising the amino acid sequence of SEQ ID NO: 14.
   54. The method of any one of embodiments 1-53, wherein the first binding arm of the bispecific antibody is derived from a humanized antibody, preferably from a full-length IgG1,λ (lambda) antibody.
   55. The method of embodiment 54, wherein the first binding arm of the bispecific antibody comprises a λ light chain constant region comprising the amino acid sequence set forth in SEQ ID NO: 22.
   56. The method of any one of embodiments 1-55, wherein the second binding arm of the bispecific antibody is derived from a human antibody, preferably from a full-length IgG1,κ (kappa) antibody.
   57. The method of embodiment 56, wherein the second binding arm comprises a K light chain constant region comprising the amino acid sequence set forth in SEQ ID NO: 23.
   58. The method of any one of embodiments 1-57, wherein the bispecific antibody is a full-length antibody with a human IgG1 constant region.
   59. The method of any one of embodiments 1-58, wherein the bispecific antibody comprises an inert Fc region.
   60. The method of any one of embodiments 1-59, wherein the bispecific antibody comprises a first heavy chain and a second heavy chain, wherein in both the first and second heavy chains, the amino acids in the positions corresponding to positions L234, L235, and D265 in the human IgG1 heavy chain constant region of SEQ ID NO: 15 are F, E, and A, respectively.
   61. The method of any one of embodiments 1-60, wherein the bispecific antibody comprises a first heavy chain and a second heavy chain, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.
   62. The method of any one of embodiments 1-61, wherein the bispecific antibody comprises a first heavy chain and a second heavy chain, wherein

(i) in both the first and second heavy chains, the amino acids in the positions corresponding to positions L234, L235, and D265 in the human IgG1 heavy chain constant region of SEQ ID NO: 15 are F, E, and A, respectively, and

(ii) in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.

63. The method of embodiment 62, wherein the bispecific antibody comprises heavy chain constant regions comprising the amino acid sequences of SEQ ID NOs: 19 and 20.
64. The method of any one of embodiments 1-63, wherein the bispecific antibody comprises a heavy chain and a light chain comprising the amino acid sequences set forth in SEQ ID NOs: 24 and 25, respectively, and a heavy chain and a light chain comprising the amino acid sequences set forth in SEQ ID NOs: 26 and 27, respectively.
65. The method of any one of embodiments 1-64, wherein the bispecific antibody comprises a heavy chain and a light chain consisting of the amino acid sequence of SEQ ID NOs: 24 and 25, respectively, and a heavy chain and a light chain consisting of the amino acid sequence of SEQ ID NOs: 26 and 27, respectively.
66. The method of any one of embodiments 1-65, wherein the bispecific antibody is epcoritamab, or a biosimilar thereof.

### EXAMPLES

### DuoBody®-CD3×CD20

DuoBody®-CD3×CD20 is a bsAb recognizing the T-cell antigen CD3 and the B-cell antigen CD20. DuoBody®-CD3×CD20 triggers potent T-cell-mediated killing of CD20-expressing cells. DuoBody®-CD3×CD20 has a regular IgG1 structure.

Two parental antibodies, IgG1-CD3-FEAL, a humanized IgG1λ, CD3ε-specific antibody having heavy and light chain sequences as listed in SEQ ID NOs: 24 and 25, respectively, and IgG1-CD20-FEAR, derived from human IgG1κ CD20-specific antibody 7D8 having heavy and light chain sequences as listed in SEQ ID NOs: 26 and 27, respectively, were manufactured as separate biological intermediates. Each parental antibody contains one of the complementary mutations in the CH3 domain required for the generation of DuoBody® molecules (F405L and K409R, respectively). The parental antibodies comprised three additional mutations in the Fc region (L234F, L235E and D265A; FEA). The parental antibodies were produced in mammalian Chinese hamster ovary (CHO) cell lines using standard suspension cell cultivation and purification technologies. DuoBody®-CD3×CD20 was subsequently manufactured by a controlled Fab-arm exchange (cFAE) process (Labrijn et al. 2013, Labrijn et al. 2014, Gramer et al. 2013). The parental antibodies are mixed and subjected to controlled reducing conditions. This leads to separation of the parental antibodies that, under re-oxidation, re-assemble. This way, highly pure preparations of DuoBody®-CD3×CD20 (— 93-95%) were obtained. After further polishing/purification, final product was obtained, close to 100% pure. The DuoBody®-CD3×CD20 concentration was measured by absorbance at 280 nm, using the theoretical extinction coefficient ε=1.597 mL·mg⁻¹ cm⁻¹. The final product was stored at 4° C. The product has an international proprietary name of epcoritamab.

Epcoritamab is prepared (5 mg/mL or 60 mg/mL) as a sterile clear colorless to slightly yellow solution supplied as concentrate for solution for subcutaneous (SC) injection. Epcoritamab contains buffering and tonicifying agents. All excipients and amounts thereof in the formulated product are pharmaceutically acceptable for subcutaneous injection products. Appropriate doses are reconstituted to a volume of about 1 mL for subcutaneous injection.

### Example 1: Impact of Lenalidomide on T-Cell Mediated Cytotoxicity Induced by Epcoritamab In Vitro

This experiment was performed to determine the impact of lenalidomide on Duobody®-CD3×CD20-induced T-cell activation and T-cell-mediated cytotoxicity.

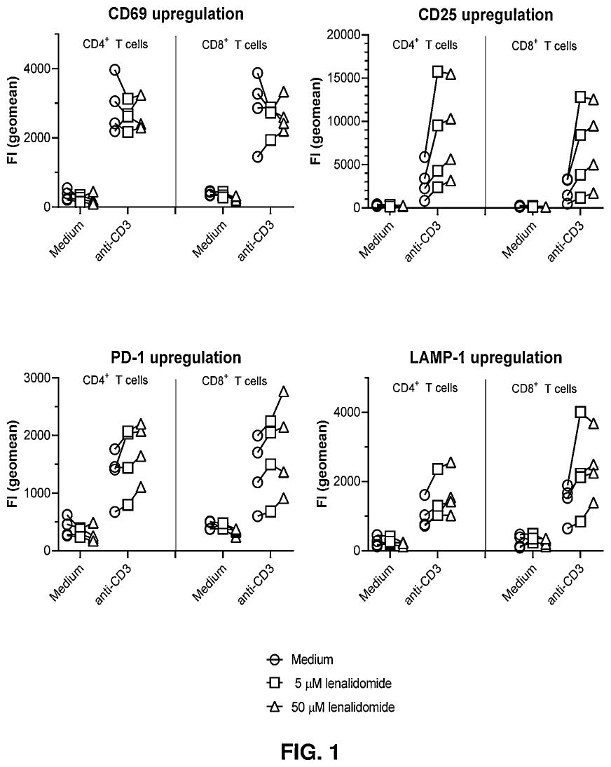

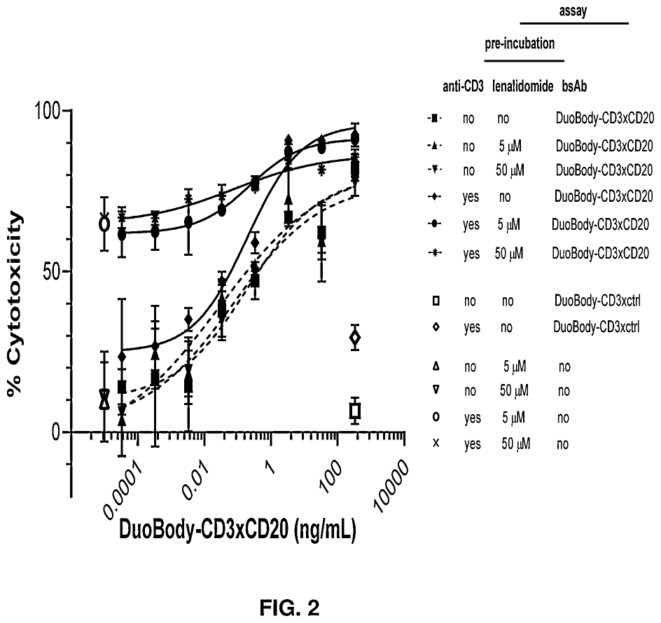

Briefly, T cells were activated with immobilized anti-CD3 in the presence (5 or 50 μM) or absence of lenalidomide for 3 days. Crosslinking of CD3 on T cells in the presence of lenalidomide led to increased T-cell activation, measured by the upregulation of activation markers CD69, CD25 on the T cell surface, and release of granzyme B and IFNγ, compared to conditions where lenalidomide was not present (see FIG. 1). T cells that were activated in the presence or absence of lenalidomide were subsequently tested for their cytotoxic capacity in response to epcoritamab. Higher maximum percent cytotoxicity and higher activity at lower epcoritamab concentrations were observed with T cells pre-treated with lenalidomide and anti-CD3 in a typical dose response curve, while none of the controls led to additional target cell lysis (See FIG. 2).

This indicates that lenalidomide may enhance the T-cell activation by epcoritamab observed in patients, which in turn can lead to more efficient T cell-mediated cytotoxicity against the target cells.

### Example 2: Phase Lb/2, Open Label Study to Evaluate Safety and Tolerability of Epcoritamab in Combination with Lenalidomide and Ibrutinib in Subjects with Diffuse Large B-Cell Lymphoma

A Phase 1b/2, open-label, multi-national, multi-center interventional trial evaluating the safety, tolerability, and preliminary efficacy of epcoritamab in combination with lenalidomide or in combination with lenalidomide and ibrutinib, in subjects diagnosed with DLBCL. The study will include a dose escalation phase followed by an expansion phase.

Summary of Ongoing Clinical Trial with Epcoritamab

Epcoritamab as monotherapy is currently in a clinical trial for the treatment of R/R B-NHL (ClinicalTrials.gov Identifier: NCT03625037).

The Phase 1 study evaluating SC epcoritamab monotherapy included subjects with R/R NHL including DLBCL. The dose escalation part of the study evaluated a range of doses (12-60 mg). A full dose of 48 mg was selected as the RP2D, following one weekly priming dose of 0.16 mg and one weekly intermediate dose of 0.8 mg.

The Phase 2 study included subjects with R/R NHL, including DLBCL, and treatment-naïve DLBCL evaluated at 24 mg and 48 mg doses in an escalation phase. Clinically meaningful and compelling efficacy with epcoritamab was seen among patients with relapsed or refractory (R/R) B-NHL in the phase 1/2 trial (NCT03625037) trial, including deep and durable responses (overall response rate (ORR), 63%; complete response (CR] rate, 39%; median duration of response (DOR), 12 months) in a population with highly refractory large B-cell lymphoma with a manageable safety profile (n=157)(J. Clin. Oncol. Dec. 22, 2022: DOI https://doi.org/10.1200/JCO.22.01725).

Combination therapy with Epcoritamab and rituximab+lenalidomide (R2) is under investigationiIn an ongoing phase 1/2 study (NCT04663347). Combination treatment with subcutaneous epcoritamab+R² led to a high CMR rate in patients with R/R FL: 73% (30/41) of patients achieving a complete metabolic response (CMR). CRS events were all grade 1 (27%) or grade 2 (10%) and were mostly associated with the first full dose, were low grade, and resolved. No new safety signals were detected (Falchi et al, Blood (2022) 140 (Supplement 1): 1464-1466).

In high-risk patients with newly diagnosed DLBCL, patients with previously untreated FL grade 1-3A who met GELF criteria received epcoritamab 48 mg+R² for 12 C of 28 days. Epcoritamab was administered QW in C1-2, and Q4W+, for up to 2 years. Step-up dosing and corticosteroid prophylaxis were required during C1 to mitigate cytokine release syndrome (CRS). As of Jun. 10, 2022, 41 patients had received treatment. Median age was 57 years (range, 39-78), and median time from initial diagnosis to first dose was 12 weeks (range, 2-352); most patients (85%) had grade 2/3A FL, 90% had stage III/IV disease, and 34% had FLIPI 3-5. At a median follow-up of 4.4 months (range, 0.7-7.5), 88% of patients remained on treatment. The most common treatment-emergent adverse events (TEAEs) were CRS (51%; 34% grade 1, 17% grade 2), neutropenia, pyrexia, injection-site reactions, fatigue, headache, constipation, and rash. Most CRS events occurred after the first full dose and all resolved in a median of 4 days. No cases of ICANS or clinical tumor lysis syndrome were observed. There was one fatal TEAE: COVID-19 pneumonia (not related to epcoritamab). In efficacy-evaluable patients (n=29), the overall response rate and complete metabolic response rate were 90% and 69%. All responses were ongoing at data cutoff.

From this study it was concluded that epcoritamab+R² demonstrated a manageable safety profile in patients with newly diagnosed DLBCL, similar to that of patients with R/R disease. CRS events were low grade and occurred around the time of first full dose.

### Objectives and Endpoints

### Primary Objectives

To characterize the safety and toxicity profiles of epcoritamab when co-administered with lenalidomide or lenalidomide and ibrutinib, in subjects with DLBCL.
To determine the recommended dose for further investigation of epcoritamab when co-administered with lenalidomide or lenalidomide and ibrutinib in subjects with DLBCL.

### Secondary Objectives

To evaluate the anti-NHL activity of epcoritamab when given in combination with lenalidomide or lenalidomide and ibrutinib, in subjects with DLBCL.
To characterize the pharmacokinetics of epcoritamab when given in combination with lenalidomide or lenalidomide and ibrutinib, in subjects with DLBCL.

### Exploratory Objectives

To evaluate potential mechanisms of response or resistance to therapy
To evaluate the immunogenicity of epcoritamab
To evaluate the impact on patient quality of life (QOL) through Patient-Reported Outcome Instruments (PRO), Functional Assessment of Cancer Therapy—Lymphoma (FACT-Lym) and EuroQol 5 Dimensions 5 Levels (EQ-5D-5L)

### Primary Endpoint

The primary endpoint is dose limiting toxicities (DLTs) of epcoritamab in combination with lenalidomide or in combination with lenalidomide and ibrutinib.

### Secondary Endpoints

Overall Response Rate (ORR) by Lugano 2014 criteria as assessed by investigator for epcoritamab in combination with lenalidomide or lenalidomide and ibrutinib.
Anti-lymphoma activity of epcoritamab in combination with lenalidomide or lenalidomide and ibrutinib:
   Duration of response (DOR) determined per Lugano 2014 criteria as assessed by investigator
   Progression free survival (PFS) determined per Lugano 2014 criteria as assessed by investigator
   Complete Response (CR) rate determined per Lugano 2014 criteria as assessed by investigator
   Time to response (TTR) determined per Lugano 2014 criteria as assessed by investigator
   Time to next anti-lymphoma therapy (TTNT)
   Rate and duration of Minimal Residual Disease (MRD) negativity
   Overall survival (OS)

### Safety Endpoints

Safety and tolerability evaluations for the duration of the study include, but are not limited to:
Monitoring severity and incidence of adverse events (AE) including adverse events of special interest (AESIs)
   Cytokine release syndrome (CRS), immune cell-associated neurotoxicity syndrome (ICANS), and clinical tumor lysis syndrome (CTLS)
Clinical laboratory testing (hematology, chemistry, and urinalysis)
   Monitoring incidence and severity of changes in laboratory values
Physical examinations
Vital signs measurements
Electrocardiogram (ECG) variables

### Pharmacokinetic Endpoints

Values for pharmacokinetic (PK) parameters, including the maximum observed plasma concentration (Cₘₐₓ), the time to Cₘₐₓ (Tₘₐₓ), and the area under the plasma concentration versus time curve (AUC) will be determined using noncompartmental methods for epcoritamab in combination with lenalidomide or lenalidomide and ibrutinib.
Epcoritamab anti-drug antibodies (ADAs) and neutralizing ADAs in combination with lenalidomide or lenalidomide and ibrutinib.

### Study Design Overview

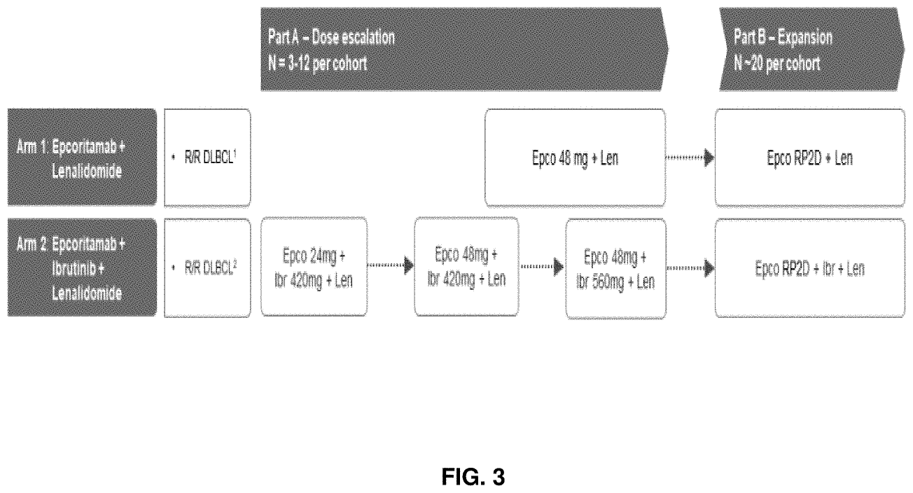

A schematic of the overall trial design is shown in FIG. 3.

### Study Arms

The following regimens will initially be evaluated in the corresponding populations:
Arm 1: epcoritamab in combination with lenalidomide in subjects with R/R DLBCL
Arm 2: epcoritamab in combination with ibrutinib and lenalidomide in subjects with R/R DLBCL

### Study Treatments

Arm 1: 12 cycles of epcoritamab in combination with Lenalidomide
Lenalidomide 25 mg oral (PO) will be administered on Days 1 through 21 (Days 22 to 28 off) of Cycles 1 to 12
Epcoritamab will be administered as noted below for 28-day cycle dosing for a total of 12 cycles
   Arm 2: 24 cycles of epcoritamab in combination with lenalidomide and Ibrutinib
Ibrutinib, 420 mg or 560 mg, will be administered orally on Days 1-28 of Cycles 1-24
Lenalidomide 20 mg will be administered orally on Days 1-21 of Cycles 1-24
Epcoritamab will be administered as noted above for 28-day cycle dosing for a total of 24 cycles
   Arm 1 and Arm 2: Epcoritamab in combination with study drug(s) will be administered using a step-up dosing method: priming dose of 0.16 mg (Cycle 1 Day 1), followed by an intermediate dose of 0.8 mg (Cycle 1 Day 8), and full doses of the assigned dose level, 24 or 48 mg (Cycle 1 Day 15 onwards). Epcoritamab will be administered as a SC injection once every week (QW) in Cycles 2-3, followed by once every 4 weeks (Q4W) in Cycle 4 through Cycle 12 Arm 1) or in Cycle 4 through Cycle 24 (Arm 2).

Each arm will consist of 2 phases: Dose Escalation (n up to 12 subjects for each dose level) and Expansion (n up to 20 subjects). Within each arm, subjects can only participate in one phase. Dose Escalation and Expansion phases of each arm will consist of a screening period, a treatment period, a post treatment follow-up period, safety follow-up period, and survival follow-up period.

### Dose Escalation Phase

The dose escalation phase is designed to assess the initial safety and tolerability of epcoritamab in combination with lenalidomide or with lenalidomide or ibrutinib.

Dose escalation will be guided by a Bayesian optimal interval (BOIN) design. For each arm, the initial enrollment in a dose escalation cohort will consist of at least 3 DLT-evaluable subjects. For each arm, epcoritamab will initially be administered in combination with the corresponding anti-neoplastic agent. Arm 1 which will begin with epcoritamab dose level 48 mg. Arm 2 will begin with epcoritamab dose level 24 mg, If acceptable safety and tolerability are observed during the DLT period, the dose of epcoritamab will be escalated to the next dose level 48 mg. The decision to de-escalate or escalate to the higher dose of epcoritamab will be made according to the BOIN design and based on the cumulative number of subjects who experience a dose limiting toxicity (DLT).

For Arm 2, the initial dose level of ibrutinib will be 420 mg. Escalation to an ibrutinib dose of 560 mg may be explored if permitted by the escalation decision rule. Only 1 agent (either epcoritamab or ibrutinib) may be escalated in a single cohort (i.e., a single cohort may not escalate both agents simultaneously).

Table 2 below provides the escalation decision rule for the BOIN design with target toxicity rate of 0.25 and optimal interval of (0.204, 0.304).

TABLE 2
Dose Escalation Decision Rule

| | # Evaluable Subjects at Current Combination | | | | | | | | | |
|---|---|---|---|---|---|---|---|---|---|---|
| Action | 3 | 4 | 5 | 6 | 7 | 8 | 9 | 10 | 11 | 12 |
| Escalate if # subjects | 0 | 0 | 1 | 1 | 1 | 1 | 1 | 2 | 2 | 2 |
| with DLT≤ | | | | | | | | | | |
| Stay at current dose if # | 1^{a} | 1 | — | — | 2 | 2 | 2 | 3 | 3 | 3 |
| subjects with DLT= | | | | | | | | | | |
| De-escalate if # subjects | 2 | 2 | 2 | 2 | 3 | 3 | 3 | 4 | 4 | 4 |
| with DLT≥ | | | | | | | | | | |
| Eliminate if # subjects | 3 | 3 | 3 | 4 | 4 | 4 | 5 | 5 | 6 | 6 |
| with DLT≥ | | | | | | | | | | |

^{a}Modified to be consistent with 3 + 3 decision rule

Dose limiting toxicities (DLTs) will be assessed during each dose-escalation cohort in order to define the recommended phase 2 dose (RP2D). For this study, the DLT evaluation period is defined as the first four weeks, i.e., 28 days after the first administration of epcoritamab.

After all subjects on a dose level have completed the DLT evaluation period, all available data will be evaluated to make a recommendation for the next dose level.

After completion of the Dose Escalation Phase, the Sponsor will review the cumulative study data and recommend a dose to be declared as the dose of epcoritamab to be used in the Dose Expansion Phase. The totality of data including safety (i.e., AEs and safety laboratory values, and observations made after the end of the DLT evaluation period), pharmacokinetics, pharmacodynamics, and preliminary efficacy will be evaluated to guide further development in the expansion phase.

### Expansion Phase

The purpose of the expansion phase is to evaluate the safety, tolerability, and preliminary clinical activity of recommended dose of epcoritamab in combination with anti-neoplastic agents.

In the expansion phase of the study, a total of approximately 20 subjects will be enrolled in each arm. Epcoritamab will be administered at the determined recommended Phase 2 dose (RP2D) in combination with lenalidomide, or lenalidomide and ibrutinib, in the same manner as was done in Dose Escalation.

A toxicity monitoring rule will be implemented in each expansion cohort after 6 subjects have been enrolled. The rule will monitor the occurrence of DLTs in each expansion cohort and will pause enrollment to a cohort if the posterior probability that the DLT rate exceeds 0.25 is greater than 80%. The prior distribution for the DLT rate in each expansion cohort will be assumed to follow a beta (1.5, 4.5) distribution, reflecting a prior mean DLT rate of 0.25 and effective sample size of 6. This corresponds to the target toxicity rate (0.25) defined in the dose escalation portion and the minimum number of subjects (6) to be enrolled at the preliminary recommended dose and schedule identified for further investigation during dose escalation.

If the number of expansion cohort subjects experiencing a DLT exceeds the toxicity boundaries at any time after 6 subjects are enrolled, subsequent enrollment to that cohort will be paused and an aggregate safety review of all available data will be performed. Based on the toxicity monitoring rule, enrollment to an expansion cohort will be paused if the number of subjects experiencing a DLT meets any of the following boundaries:
≥3 subjects of 6 subjects enrolled
≥4 subjects of 7 to 9 subjects enrolled
≥5 subjects of 10 to 12 subjects enrolled
≥6 subjects of 13 to 16 subjects enrolled
≥7 subjects of 17 to 19 subjects enrolled
≥8 subjects of 20 subjects enrolled

### Inclusion Criteria

Subjects must meet all of the following criteria in order to be included in the study:
Adult male or female, at least 18 years old.
Laboratory values meeting the following criteria within the screening period prior to the first dose of study drug:
   Absolute neutrophil count (ANC)≥1.0×109/L (growth factor use is allowed if evidence of bone marrow involvement, but subject must not have received growth factor within 14 days prior to screening labs)
   Hemoglobin ≥8.0 g/dL (RBC transfusions permitted, but subject must not have received blood transfusions within 7 days prior to screening labs)
   Platelet count ≥75×109/L, or ≥50×109/L if bone marrow infiltration or splenomegaly (platelet transfusions permitted, but subject must not have received blood transfusions within 7 days prior to screening labs)
   Serum aspartate transaminase (AST) or alanine transaminase (ALT) level ≤3×ULN
   Total bilirubin level ≤1.5×ULN or ≤5×ULN for subjects with hepatic involvement of disease or of non-hepatic origin. Subjects with Gilbert's syndrome may have total bilirubin levels >1.5×ULN, but direct bilirubin must be <2×ULN
   Estimated Creatinine Clearance (CrCl)≥50 mL/min (as calculated by Cockcroft-Gault Formula, modified as needed for factors such as body weight)
   Prothrombin time (PT)/International normalized ratio (INR)/Activated partial thromboplastin time (aPTT)≤1.5×ULN, unless receiving anticoagulation
Subject must be able to tolerate subcutaneous injections
Subject must have available adequate fresh or paraffin-imbedded tissue at Screening

### Disease/Condition Activity

Diagnosis of DLBCL (de novo or histologically transformed from follicular lymphoma or nodal marginal zone lymphoma) with histologically confirmed CD20+ disease, inclusive of the following according to WHO 2016 classification and documented in pathology report:
   DLBCL, not otherwise specified (NOS)
   High-grade B cell lymphoma with MYC and BCL-2 and/or BCL-6 translocations per WHO 2016 (“double-hit” or “triple-hit”)
   Note: High-grade B-cell lymphomas NOS or other double-/triple-hit lymphomas (with histologies not consistent with DLBCL) are not eligible
   Follicular lymphoma Grade 3B
Subject must have no prior treatment with a bispecific antibody targeting CD3 and CD20
Subject must have 1 or more measurable disease sites:
A positron emission tomography/computed tomography (PET/CT) scan demonstrating PET-positive lesion(s)
AND
At least 1 measurable nodal lesion (long axis ≥1.5 cm and short axis >1.0 cm) or ≥1 measurable extra-nodal lesion (long axis ≥1.0 cm) on CT scan or MRI
Subject must be eligible to receive and have a need for treatment initiation based on symptoms and/or disease burden as per investigator assessment.
Subject must have Eastern Cooperative Oncology Group (ECOG) performance status 0-2.
Subject has no unresolved toxicities from prior anticancer therapy, defined as having not resolved to Common Terminology Criteria for Adverse Events (CTCAE, v 5.0), Grade 1, with the exception of alopecia. Other eligibility criteria (e.g., laboratory, cardiac criteria) must also be met.
Subject has no current evidence of primary central nervous system (CNS) tumor or known
CNS involvement, including leptomeningeal disease, at screening.
Subject has no history of severe allergic or anaphylactic reactions to anti-CD20 mAb therapy or known significant allergy or intolerance to any component or excipient of epcoritamab or components of study drug combination agents (e.g., lenalidomide, ibrutinib, etc.)
Subject must not have had autologous stem cell transplantation within 3 months prior to screening.
Subject must not have had chemotherapy, non-investigational, or investigational anti-neoplastic agents (except CD20 mAbs) within 4 weeks or 5 half-lives (whichever is shorter) prior to the first dose of epcoritamab.
Subject has no clinically significant cardiovascular disease, including:
   Myocardial infarction or stroke within 6 months prior to enrollment,
   OR
   The following conditions within 3 months prior to enrollment: unstable or uncontrolled disease/condition related to or affecting cardiac function (e.g., unstable angina, congestive heart failure, New York Heart Association Class III-IV), uncontrolled cardiac arrhythmia
   OR
   Other clinically significant electrocardiogram (ECG) abnormalities within 6 months prior to enrollment unless deemed stable and appropriately treated
   OR
   Left ventricular ejection fraction <45%.
Subject has no clinically significant liver disease, including hepatitis, current alcohol abuse, or cirrhosis.
Subject does not have active Hepatitis B Virus (HBV) or Hepatitis C Virus (HCV) infection.
Subjects who are positive for hepatitis B core antibody (HBcAb), hepatitis B surface antigen (HBsAg), or hepatitis C antibody must have a negative polymerase chain reaction (PCR) result before enrollment. Those who are PCR positive will be excluded.
Subject has no known history of Human Immunodeficiency Virus (HIV) infection. Note: HIV testing does not need to be conducted at screening unless it is required per local guidelines or institutional standards.
Subject has no known active bacterial, viral, fungal, mycobacterial, parasitic, or other infection (excluding fungal infections of the nail beds) requiring intravenous (IV) therapy or
IV antibiotics within 2 weeks prior to enrollment.
Subject has no evidence of significant, uncontrolled concomitant diseases that could affect compliance with the protocol or interpretation of results.
Subject has no history of other prior malignancies, except for the following:
   Malignancy treated with curative intent and with no known active disease present for ≥3 years before the first dose of study drug and felt to be at low risk for recurrence by the treating physician
   Adequately treated non-melanoma skin cancer or lentigo maligna without evidence of disease
   Adequately treated carcinoma in situ without evidence of disease
   Localized prostate cancer, post-radical prostatectomy with non-rising prostate-specific antigen (PSA) levels <0.1 ng/mL
Subject has not had radiation therapy to target lesion if only 1 target lesion is involved and no other target lesions that have not received radiation therapy can be followed, or major surgery within 4 weeks of enrollment.
Subject has no Grade >1 neuropathy.
Subject must not have active tuberculosis (TB) or history of completed treatment for active TB within the past 12 months.
   Note: Interferon gamma release assay (IGRA) testing does not need to be performed at screening unless active or latent tuberculosis is suspected. For subjects with positive IGRA, active pulmonary tuberculosis must be excluded with clinical evaluation and radiologic imaging. Subjects with positive IGRA and no evidence of active disease may be enrolled after treatment for latent tuberculosis infection (recommendation isoniazid monotherapy for total of 6 months) has been initiated.
Subject has no evidence of cytomegalovirus (CMV) viremia (defined as any positive level above the lower limit of detection) at screening.
Subject has no current autoimmune disease requiring immunosuppressive therapy except for up to 20 mg prednisone daily (or equivalent).
Subject has no life-threatening illness, medical condition, or organ system dysfunction that, in the Investigator's opinion, could compromise the subject's safety or put the study outcomes at undue risk.
Subject has no current seizure disorder requiring therapy.
Subject has no known active SARS-CoV-2 infection. If a subject has signs/symptoms suggestive of SARS-CoV-2 infection or have recent known exposure to someone with SARS-CoV infection, they should undergo molecular (e.g., PCR) testing or 2 negative antigen test results at least 24 hours apart to rule out SARS-CoV-2 infection.
Subjects who do not meet SARS-CoV-2 infection eligibility criteria must be screen failed and may only rescreen after they meet the following SARS-CoV-2 infection viral clearance criteria:
   At least 10 days since first positive test result have passed in asymptomatic patients or at least 10 days since recovery, defined as resolution of fever without use of antipyretics and improvement in symptoms.
   Subject must not have had major surgery within 4 weeks of the first dose of study drug.

### Additional Eligibility Criteria Specific to Arm 1

Subject must have relapsed/refractory DLBCL
Note: Relapsed disease is defined as disease that previously responded to therapy but progressed ≥6 months after completion of therapy. Refractory disease is defined as disease that either progressed during therapy, failed to achieve an objective response to prior therapy, or progressed within 6 months after completion of therapy (including maintenance therapy).
Subject must have R/R disease to at least one prior systemic anti-lymphoma therapy (radiotherapy is not considered a systemic therapy) which contains an anti-CD20 monoclonal antibody. Subject who received only prior anti-CD20 monoclonal antibody monotherapy is not eligible.
Subject must not be refractory (defined as best response of SD or PD) to prior CAR-T therapy.
Subjects must have either failed prior autologous stem cell transplant (ASCT), not be considered eligible for ASCT due to age, performance status, comorbidities and/or insufficient response, or have refused ASCT.
Subject must not have documented refractoriness to lenalidomide and must be suitable for treatment with lenalidomide in the opinion of the investigator.
Note: Refractoriness is defined as:
   Best response to prior regimen(s) of stable disease (SD) or progressive disease (PD),
   OR
   Progressive disease within 6 months of completion of prior regimen(s)
Subject must be willing to take aspirin prophylaxis or prophylactic anticoagulation for thromboembolic event (or per local guidelines for lenalidomide administration).
Female subjects of childbearing potential must practice at least 2 protocol-specified methods of birth control that are effective from 30 days prior to enrollment through at least 12 months after the last dose of study drug. Female subjects of non-childbearing potential do not need to use birth control.
Subject is willing to adhere to the pregnancy risk minimization plan associated with lenalidomide treatment.
Subject must not have had lenalidomide exposure within 12 months prior to screening.

### Additional Eligibility Criteria Specific to Arm 2:

Subject must have R/R DLBCL (see above for definition).
Subject must have received at least 1 prior treatment for which must include an anti-CD20 monoclonal antibody in combination with another systemic therapy.
Subject must have received prior CAR-T cell therapy, but for those who achieved a response to prior CAR-T, not less than 90 days prior to first dose of epcoritamab, or for those who were refractory to CAR-T, not less than 60 days prior to first dose of epcoritamab.
Note: Refractoriness is defined as:
   Best response to prior regimen(s) of stable disease (SD) or progressive disease (PD),
   OR
   Progressive disease within 6 months of completion of prior regimen(s)
Subjects must have either failed prior ASCT, not be considered eligible for ASCT due to age, performance status, comorbidities and/or insufficient response, or have refused ASCT
Subject must not have documented refractoriness to lenalidomide and must be suitable for treatment with lenalidomide in the opinion of the investigator.
Subject must not have had prior treatment with ibrutinib and must be suitable for treatment with ibrutinib in the opinion of the investigator.
Subject must not have known bleeding diathesis (e.g., von Willebrand's disease) or hemophilia.
Subject must not require treatment with a strong cytochrome P450 (CYP) 3A inhibitor.
Subject must be willing to take aspirin prophylaxis or prophylactic anticoagulation for thromboembolic event (or per local guidelines for lenalidomide administration).
Female subjects of childbearing potential must practice at least 2 protocol-specified methods of birth control that are effective from 30 days prior to enrollment through at least 12 months after the last dose of study drug. Female subjects of non-childbearing potential do not need to use birth control.
Subject is willing to adhere to the pregnancy risk minimization plan associated with lenalidomide treatment.
Subject must be able to swallow capsules and must not have any disease significantly affecting gastrointestinal function (e.g., resection of the stomach or small bowel, symptomatic inflammatory bowel disease, or partial or complete bowel obstruction).

### Dose-Limiting Toxicities

A DLT-evaluable subject in the dose escalation phase is defined as a subject who has received at least 3 doses of epcoritamab at the assigned dose level in the first cycle or experiences a DLT during the 28-day period after the first dose of epcoritamab.

The DLT evaluation period is defined as the first 4 weeks, i.e., 28 days after the first administration of epcoritamab, provided the subject has received at least 3 epcoritamab doses during this period.

The following will qualify for a DLT, unless the Investigator can attribute the event to a clearly identifiable cause such as underlying illness, disease progression/relapse, other concurrent illness, or from concomitant therapy.
Grade 5 toxicity
CRS grading according to American Society for Transplantation and Cellular Therapy (ASTCT) criteria and DLT criteria for CRS
   Grade 4 CRS or ICANS according to ASTCT criteria
   Grade 3 CRS or ICANS according to ASTCT criteria which has NOT improved to
   Grade ≤2 or resolved (Grade 0) within 48 hours
Neutropenia Grade 4 lasting >7 days Graded by CTCAE.
Febrile neutropenia Grade ≥3 lasting >2 days Graded by CTCAE.
Thrombocytopenia Grade 4 lasting >7 days Graded by CTCAE.
Non-hematological toxicity Grade 3 or higher as Graded by CTCAE, excluding the following:
   Grade 3 fever (>40.0° C. for ≤24 hours)
   Grade 3 hypotension (resolving within 24 hours)
   Laboratory values out of normal range which do not have any clinical consequence, are clinically transient, isolated in nature and which resolve within 7 days (this includes electrolyte abnormalities that respond to medical intervention)
   AST and/or ALT Grade 3 returned to Grade 1 or baseline within 7 days.
   Grade 3 nausea that responds to optimal antiemetic treatment within 3 days.
   Grade 3 vomiting that responds to optimal antiemetic treatment within 3 days.
   Grade 3 diarrhea that responds to optimal antidiarrheal treatment within 3 days.
   Grade 3 fatigue/asthenia when fatigue/asthenia was present at baseline or that lasts for <14 days after the last administration of epcoritamab.
   Other Grade 3 toxicity related to prior chemotherapy that was present at baseline (Grade 1 or 2) and returned to baseline within 7 days.
   Alopecia (no grading)

Frequent laboratory monitoring of complete blood count including differential should be initiated to document start and resolution of hematological AEs. All AEs occurring during the defined DLT evaluation period will be assessed according to the criteria above. All AEs, including those not qualifying for a DLT, will be monitored and included in the evaluation of the toxicity profile of epcoritamab unless the event is clearly determined to be unrelated to epcoritamab.

### Adverse Events of Special Interest

The following adverse events of special interest will be monitored during the study:
Cytokine Release Syndrome (CRS)
Clinical Tumor Lysis Syndrome (CTLS)
Immune Cell-Associated Neurotoxicity Syndrome (ICANS)

### CRS Prophylaxis and Premedication

Premedication with corticosteroids, antihistamines, and antipyretics is mandatory as described in the Operations Manual, Section 3.4. For the first four doses of epcoritamab, premedication with antihistamines, antipyretics, and corticosteroids are mandatory; and an additional 3 days of corticosteroids are required following each of these first 4 doses to prevent/reduce the severity of symptoms from potential CRS. For the first 4 doses of epcoritamab, the subject must perform self-administered oral temperature monitoring 3 times a day (approximately every 6-8 hours during waking hours) for the first 4 days post epcoritamab administration. These temperature checks are to ensure that fever, an early sign of CRS, has not developed. For administration of epcoritamab beyond the fourth dose (i.e., the second full dose), CRS prophylaxis with corticosteroids is optional, unless CRS Grade 2 or higher occurs, in which case CRS prophylaxis should continue until an epcoritamab dose is given without subsequent CRS. Premedication corticosteroid administration can be either IV or PO with the recommended dose or equivalent.

### Study Assessments

### Disease Response and Progressive Disease Assessment

On-treatment assessment: Response at on-treatment time-points should be read according to Lugano Classification for patients showing CR, PR, and SD. For patients showing PD according to Lugano Classification, further evaluation should be performed to see if the subject can be considered to have IR (according to LYRIC).

### Lugano Response Criteria for Malignant Lymphoma

### Target and Non-Target Lesions

Target lesions should consist of up to six of the largest dominant nodes, nodal masses, or other lymphomatous lesions that are measurable in two diameters and should preferably be from different body regions representative of the subject's overall disease burden, including mediastinal and retroperitoneal disease, where applicable. At baseline, a measurable node must be greater than 15 mm in longest diameter (longest transverse diameter of a lesion; LDi). Measurable extranodal disease may be included in the six representative target lesions. At baseline, measurable extranodal lesions should be greater than 10 mm in LDi.

All other lesions (including nodal, extra-nodal, and assessable disease) should be followed as non-target lesions (e.g., cutaneous, GI, bone, spleen, liver, kidneys, pleural or pericardial effusions, ascites, bone, bone marrow).

### Split Lesions and Confluent Lesions

Lesions may split or may become confluent over time. In the case of split lesions, the individual product of the perpendicular diameters (PPDs) of the nodes should be summed together to represent the PPD of the split lesion; this PPD is added to the sum of the PPDs of the remaining lesions to measure response. If subsequent growth of any or all of these discrete nodes occurs, the nadir of each individual node is used to determine progression. In the case of confluent lesions, the PPD of the confluent mass should be compared with the sum of the PPDs of the individual nodes, with more than 50% increase in PPD of the confluent mass compared with the sum of individual nodes necessary to indicate PD. The LDi and smallest diameter (shortest axis perpendicular to the LDi; SDi) are no longer needed to determine progression.

TABLE 3
Lugano Response Criteria for Malignant Lymphoma

| | | | CT-Based Response |
|---|---|---|---|
| | | | Complete Radiologic |
| | | PET-CT-Based Response | Response (all of the |
| Response | Site | Complete Metabolic Response | following) |
| Complete | Lymph nodes | Score 1, 2, or 3¹ with or without a | Target nodes/nodal masses |
| Response | and | residual mass on 5PS². | must regress to ≤1.5 cm |
| | extralymphatic | It is recognized that in Waldeyer's | in LDi. No extralymphatic |
| | sites | ring or extranodal sites with high | sites of disease |
| | | physiologic uptake or with | |
| | | activation within spleen or | |
| | | marrow (e.g., with chemotherapy | |
| | | or myeloid colony-stimulating | |
| | | factors), uptake may be greater | |
| | | than normal mediastinum and/or | |
| | | liver. In this circumstance, | |
| | | complete metabolic response may | |
| | | be inferred if uptake at sites of | |
| | | initial involvement is no greater | |
| | | than surrounding normal tissue | |
| | | even if the tissue has high | |
| | | physiologic uptake. | |
| | Non-measured | Not applicable | Absent |
| | lesion | | |
| | Organ | Not applicable | Regress to normal |
| | enlargement | | |
| | New lesions | None | None |
| | Bone marrow | No evidence of FDG-avid disease | Normal by morphology; if |
| | | in marrow | indeterminate, IHC negative |
| | | | Partial Remission |
| | | Partial Metabolic Response | (all of the following) |
| Partial | Lymph nodes | Score 4 or 5² with reduced uptake | ≥50% decrease in SPD of up |
| Response | and | compared with baseline and | to 6 target measurable nodes |
| | extralymphatic | residual mass(es) of any size | and extranodal sites |
| | sites | At interim, these findings suggest | When a lesion is too small to |
| | | responding disease. At end of | measure on CT, assign |
| | | treatment, these findings indicate | 5 mm × 5 mm as the default |
| | | residual disease. | value When no longer visible, |
| | | | 0 × 0 mm. For a node >5 |
| | | | mm × 5 mm, but smaller than |
| | | | normal, use actual |
| | | | measurement for calculation |
| | Non- | Not applicable | Absent/normal, regressed, but |
| | measured | | no increase |
| | lesion | | |
| | Organ | Not applicable | Spleen must have regressed |
| | enlargement | | by >50% in length beyond |
| | | | normal |
| | New lesions | None | None |
| | Bone marrow | Residual uptake higher than | Not applicable |
| | | uptake in normal marrow but | |
| | | reduced compared with baseline | |
| | | (diffuse uptake compatible with | |
| | | reactive changes from | |
| | | chemotherapy allowed). If there | |
| | | are persistent focal changes in the | |
| | | marrow in the context of a nodal | |
| | | response, consideration should be | |
| | | given to further evaluation with | |
| | | MRI or biopsy or an interval scan. | |
| | | No metabolic response | Stable disease |
| No | Target | Score 4 or 5² with no significant | <50% decrease from baseline |
| response or | nodes/nodal | change in FDG uptake from | in SPD of up to 6 dominant, |
| Stable | masses, | baseline at interim or end of | measurable nodes and |
| disease | extranodal | treatment | extranodal sites; no criteria for |
| | lesions | | progressive disease are met |
| | Non- | Not applicable | No increase consistent with |
| | measured | | progression |
| | lesion | | |
| | Organ | Not applicable | No increase consistent with |
| | enlargement | | progression |
| | New lesions | None | None |
| | Bone marrow | No change from baseline | Not applicable |
| | | | Progressive disease requires |
| | | Progressive metabolic disease | at least 1 of the following |
| Progressive | Individual | Score 4 or 5² with an increase in | PPD progression: |
| disease | target | intensity of uptake from baseline | An individual node/lesion |
| | nodes/nodal | and/or | must be abnormal with: |
| | masses, | New FDG-avid foci consistent | LDi >1.5 cm and |
| | extranodal | with lymphoma at interim or end- | Increase by ≥50% from |
| | lesions | of-treatment assessment | PPD nadir and |
| | | | An increase in LDi or SDi |
| | | | from nadir |
| | | | 0.5 cm for lesions ≤2 cm |
| | | | 1.0 cm for lesions >2 cm |
| | | | In the setting of splenomegaly |
| | | | (>13 cm), the splenic length |
| | | | must increase by >50% of the |
| | | | extent of its prior increase |
| | | | beyond baseline (e.g., a 15-cm |
| | | | spleen must increase to ≥16 |
| | | | cm). If no prior |
| | | | splenomegaly, must increase |
| | | | by at least 2 cm from baseline. |
| | | | New or recurrent |
| | | | splenomegaly |
| | Non- | None | New or clear progression of |
| | measured | | pre-existing non-measured |
| | lesions | | lesions |
| | New lesions | New FDG-avid foci consistent | Regrowth of previously |
| | | with lymphoma rather than | resolved lesions |
| | | another etiology (e.g., infection, | A new node >1.5 cm in any |
| | | inflammation); if uncertain | axis |
| | | regarding etiology of new lesions, | A new extranodal site >1.0 cm |
| | | biopsy or interval scan may be | in any axis; if <1.0 cm in any |
| | | considered | axis, its presence must be |
| | | | unequivocal and must be |
| | | | attributable to lymphoma |
| | | | Assessable disease of any size |
| | | | unequivocally attributable to |
| | | | lymphoma |
| | Bone marrow | New or recurrent FDG-avid foci | New or recurrent involvement |

5PS = 5-point scale; CT = computed tomography; FDG = fluorodeoxyglucose;
IHC = immunohistochemistry; LDi = longest transverse diameter of a lesion; MRI = magnetic resonance imaging; PET = positron emission tomography; PPD = cross product of the LDi and perpendicular diameter; SDi = shortest axis perpendicular to the LDi; SPD = sum of the product of the perpendicular diameters for multiple lesions.
¹A score of 3 in many subjects indicates a good prognosis with standard treatment, especially if at the time of an interim scan. However, in trials involving PET where de-escalation is investigated, it may be preferable to consider a score of 3 as inadequate response (to avoid undertreatment).
Measured dominant (target) lesions: Up to six of the largest dominant nodes, nodal masses, and extranodal lesions selected to be clearly measurable in two diameters.
Nodes should preferably be from disparate regions of the body and should include, where applicable, mediastinal, and retroperitoneal areas.
Non-nodal lesions include those in solid organs (e.g., liver, spleen, kidneys, lungs), gastrointestinal involvement, cutaneous lesions, or those noted on palpation.
Non-measured lesions: Any disease not selected as measured, dominant disease and truly assessable disease should be considered not measured.
These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging.
In Waldeyer's ring or in extranodal sites (e.g., GI tract, liver, bone marrow), FDG uptake may be greater than in the mediastinum with complete metabolic response but should be no higher than surrounding normal physiologic uptake (e.g., with marrow activation as a result of chemotherapy or myeloid growth factors).
²PET 5PS: 1 = no uptake above background; 2 = uptake ≤ mediastinum; 3 = uptake > mediastinum but ≤ liver; 4 = uptake moderately > liver; 5 = uptake markedly higher than liver and/or new lesions; × = new areas of uptake unlikely to be related to lymphoma.
Source: Cheson B D, Fisher R, Barrington S F, et al. Recommendations for initial evaluation, staging, and response assessment of Hodgkin and non-Hodgkin lymphoma: the Lugano classification. J Clin Oncol. 2014; 32: 3059-68.

### Lymphoma Response to Immunomodulatory Therapy Criteria (LYRIC)

Clinical studies have shown that cancer immunotherapies may result in early apparent radiographic progression (including the appearance of new lesions), followed by a delayed response. As this initial increase in tumor size might be caused by immune-cell infiltration in the setting of a T-cell response, this progression may not be indicative of true disease progression and is therefore called “pseudoprogression”.

The association of epcoritamab (GEN3013; DuoBody®-CD3×CD20) with pseudoprogression is currently unknown, but its mechanism of action implies that pseudoprogression is to be expected.

The current Lugano response assessment criteria does not take pseudoprogression into account, and there is a significant risk of premature discontinuation of a potentially efficacious immunomodulatory drug following the observation of an atypical response. Atypical responses are characterized either by the early progression of existing lesions, later followed by response, or by the development of new lesions, with or without tumor shrinkage elsewhere.

LYRIC is a modification of the Lugano response assessment criteria, which has been adapted to immune-based therapies, and it implements a new, mitigating response category: the “indeterminate response” (IR) designation.⁵ This IR designation was introduced to potentially identify “atypical response” cases until confirmed as flare/pseudoprogression or true PD by either biopsy or subsequent imaging. LYRIC and the Lugano criteria will be assessed in this study.

### Indeterminate Response (IR) Category

A subject who shows PD according Lugano Classification will be considered to have IR in 1 or more of the 3 following circumstances.

IR (1): Increase in overall tumor burden (as assessed by sum of the product of the diameters [SPD]) of ≥50% of up to 6 target lesions in the first 12 weeks of therapy, without clinical deterioration.
IR (2): Appearance of new lesions or growth of one or more existing lesion(s)≥50% at any time during treatment; occurring in the context of lack of overall progression (SPD<50% increase) of overall tumor burden, as measured by SPD of up to 6 lesions at any time during the treatment.
IR (3): Increase in FDG uptake of 1 or more lesion(s) without a concomitant increase in lesion size or number.
Screening assessment: At screening, the FDG-PET/CT and diagnostic CT or MRI scans should be read according to Lugano Classification, as detailed above.
On-treatment assessment: Response at on-treatment time-points should be read according to Lugano Classification for patients showing CR, PR, and SD. For patients showing PD according to Lugano Classification, further evaluation should be performed to see if the subject can be considered to have IR (according to LYRIC).

### Statistical Analyses for Efficacy

Descriptive statistics and subject listings will be used to summarize the data for each epcoritamab dose level (24 mg and 48 mg). For continuous variables, number of observations, means, standard deviations, medians, and ranges will be used. For categorical variables, frequency and percentage will be summarized. For time-to-event endpoints, Kaplan-Meier estimates will be provided.

### Summary and Analysis of Key Secondary Efficacy Endpoints

Overall response rate (ORR) is defined as the proportion of subjects who achieved best overall response of CR or PR determined by Lugano 2014 criteria as assessed by investigators. Point estimate along with 95% exact confidence interval (CI) will be provided for each arm.

Duration of response (DOR) is defined for subjects who achieved best overall response of CR or PR (‘responders’), as the time in months from initial CR/PR to the earliest occurrence of disease progression determined by Lugano 2014 criteria as assessed by investigator, or death from any cause. Surviving responders without radiographic disease progression will be censored at the time of the last adequate disease assessment.

Number of responders, number of DOR events and the earliest contributing event (disease progression or death) will be summarized by arm. The Kaplan-Meier method will be used to estimate the distribution of DoR for each arm.

Progression-free survival (PFS) is defined for subjects in all arms, as the time in months from the first dose of study drug to the earliest occurrence of disease progression determined by Lugano 2014 criteria as assessed by investigator, or death from any cause. Surviving subjects without disease progression will be censored at the time of the last adequate disease assessment. Surviving subjects without post-baseline disease assessment will be censored at the date of first dose of study drug.

Number of PFS events and the earliest contributing event (disease progression or death) will be presented by arm. The Kaplan-Meier method will be used to estimate the distribution of PFS.

Complete response rate is defined as the proportion of subjects who achieved best overall response of CR determined by Lugano 2014 criteria as assessed by investigator. Point estimate along with 95% exact confidence interval (CI) will be provided for each arm.

Time to response (TTR) is defined for subjects who achieved best overall response of CR or PR (‘responders’) determined by Lugano 2014 criteria as assessed by investigator, as the time in months from first dose of study drug to initial CR/PR.

Number of responders along with descriptive summaries of TTR will be provided for each arm.

Overall survival (OS) is defined for subjects in all arms, as the time in months from first dose of epcoritamab to death from any cause. Subjects that are still alive at the end of the study or at the time of the analysis will be censored at last known alive date.

Number of deaths, and Kaplan-Meier estimates of distribution of OS will be provided.

### Statistical Analyses for Safety

Safety and tolerability of epcoritamab in combination with other agents will be assessed by evaluation of study drug exposure, incidence of dose interruptions, reductions, delays and discontinuations, AEs including AESIs, SAEs, deaths and changes in adverse events and vital signs parameters.

Treatment-emergent AEs will be summarized by Preferred Terms within a System Organ Class according to the Medical Dictionary for Regulatory Activities. The number and percentage of subjects experiencing a DLT will be summarized. Additional details will be provided in the SAP.

Where applicable, blood chemistry and hematology laboratory determinations will be categorized according to the NCI CTCAE and summarized. Additional details will be provided in the SAP.

### Statistical Analyses for Pharmacokinetics

Plasma concentrations for epcoritamab along with PK parameter values will be tabulated for drug within each cohort. Summary statistics will be computed by sampling time for PK concentrations and by cycle and/or visits for PK parameters. Results for epcoritamab ADA (and nAb, if applicable) will be summarized. Additional exploratory analyses may be conducted as deemed appropriate.

### Preliminary Results

Arm 1: Dose Escalation (Epcoritamab 24 mg+Len 25 mg):

### # of Subjects Enrolled: 5

# of Subjects projected with at least 1 post-baseline efficacy assessment: 5
# of Subjects with available post-baseline efficacy assessment: 3
ORR=100% (3/3)
CRR=100% (3/3)
   Arm 1: Dose Expansion (Epcoritamab 48 mg+Len 25 mg):

### # of Subjects Enrolled: 17

# of Subjects projected with at least 1 post-baseline efficacy assessment: 3
# of Subjects with available post-baseline efficacy assessment: 0

TABLE 4
Summary of Sequences

| SEQ | | |
|---|---|---|
| ID | Description | Sequence |
| 1 | huCD3 VH CDR1 | GFTFNTYA |
| 2 | huCD3 VH CDR2 | IRSKYNNYAT |
| 3 | huCD3 VH CDR3 | VRHGNFGNSYVSWFAY |
| 4 | huCD3 VL CDR1 | TGAVTTSNY |
| — | huCD3 VL CDR2 | GTN |
| 5 | huCD3 VL CDR3 | ALWYSNLWV |
| 6 | huCD3 VH1 | EVKLVESGGGLVQPGGSLRLSCAASGFTFNTYAMNWVRQAPGKGLE |
| | | WVARIRSKYNNYATYYADSVKDRFTISRDDSKSSLYLQMNNLKTED |
| | | TAMYYCVRHGNFGNSYVSWFAYWGQGTLVTVSS |
| 7 | huCD3 VL1 | QAVVTQEPSFSVSPGGTVTLTCRSSTGAVTTSNYANWVQQTPGQAF |
| | | RGLIGGTNKRAPGVPARFSGSLIGDKAALTITGAQADDESIYFCAL |
| | | WYSNLWVFGGGTKLTVL |
| 8 | VH CD20-7D8 CDR1 | GFTFHDYA |
| 9 | VH CD20-7D8 CDR2 | ISWNSGTI |
| 10 | VH CD20-7D8 CDR3 | AKDIQYGNYYYGMDV |
| 11 | VL CD20-7D8 CDR1 | QSVSSY |
| — | VL CD20-7D8 CDR2 | DAS |
| 12 | VL CD20-7D8 CDR3 | QQRSNWPIT |
| 13 | VH CD20-7D8 | EVQLVESGGGLVQPDRSLRLSCAASGFTFHDYAMHWVRQAPGKGLE |
| | | WVSTISWNSGTIGYADSVKGRFTISRDNAKNSLYLQMNSLRAEDTAL |
| | | YYCAKDIQYGNYYYGMDVWGQGTTVTVSS |
| 14 | VL CD20-7D8 | EIVLTQSPATLSLSPGERATLSCRASQSVSSYLAWYQQKPGQAPRLLIY |
| | | DASNRATGIPARFSGSGSGTDFTLTISSLEPEDFAVYYCQQRSNWPITF |
| | | GQGTRLEIK |
| 15 | IgG1 heavy chain | ASTKGPSVFPLAPSSKSTSGGTAALGCLVKDYFPEPVTVSWNSGALTS |
| | constant region-WT | GVHTFPAVLQSSGLYSLSSVVTVPSSSLGTQTYICNVNHKPSNTKVDK |
| | (amino acids positions | RVEPKSCDKTHTCPPCPAPELLGGPSVFLFPPKPKDTLMISRTPEVTCV |
| | 118-447 according to | VVDVSHEDPEVKFNWYVDGVEVHNAKTKPREEQYNSTYRVVSVLTV |
| | EU numbering). | LHQDWLNGKEYKCKVSNKALPAPIEKTISKAKGQPREPQVYTLPPSRE |
| | CHS region italics | EMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 16 | IgG1-LFLEDA heavy | ASTKGPSVFPLAPSSKSTSGGTAALGCLVKDYFPEPVTVSWNSGALTS |
| | chain constant region | GVHTFPAVLQSSGLYSLSSVVTVPSSSLGTQTYICNVNHKPSNTKVDK |
| | (amino acids positions | RVEPKSCDKTHTCPPCPAPEFEGGPSVFLFPPKPKDTLMISRTPEVTCV |
| | 118-447 according to | VVAVSHEDPEVKFNWYVDGVEVHNAKTKPREEQYNSTYRVVSVLTV |
| | EU numbering). | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 17 | IgG1 F405L | ASTKGPSVFPLAPSSKSTSGGTAALGCLVKDYFPEPVTVSWNSGALTS |
| | (amino acids positions | GVHTFPAVLQSSGLYSLSSVVTVPSSSLGTQTYICNVNHKPSNTKVDK |
| | 118-447 according to | RVEPKSCDKTHTCPPCPAPELLGGPSVFLFPPKPKDTLMISRTPEVTCV |
| | EU numbering) | VVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FLLYSKLTVDKSRWQQGNVFSCSVMHEALHNHYTQKSLSLSPG |
| 18 | IgG1-K409R | ASTKGPSVFPLAPSSKSTSGGTAALGCLVKDYFPEPVTVSWNSGALTS |
| | (amino acids positions | GVHTFPAVLQSSGLYSLSSVVTVPSSSLGTQTYICNVNHKPSNTKVDK |
| | 118-447 according to | RVEPKSCDKTHTCPPCPAPELLGGPSVFLFPPKPKDTLMISRTPEVTCV |
| | EU numbering) | VVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSRLTVDKSRWQQGNVFSCSVMHEALHNHYTQKSLSLSPG |
| 19 | IgG1-LFLEDA-F405L | ASTKGPSVFPLAPSSKSTSGGTAALGCLVKDYFPEPVTVSWNSGALTS |
| | (FEAL) | GVHTFPAVLQSSGLYSLSSVVTVPSSSLGTQTYICNVNHKPSNTKVDK |
| | (amino acids positions | RVEPKSCDKTHTCPPCPAPEFEGGPSVFLFPPKPKDTLMISRTPEVTCV |
| | 118-447 according to | VVAVSHEDPEVKFNWYVDGVEVHNAKTKPREEQYNSTYRVVSVLTV |
| | EU numbering) | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FLLYSKLTVDKSRWQQGNVFSCSVMHEALHNHYTQKSLSLSPG |
| 20 | IgG1-LFLEDA-K409R | ASTKGPSVFPLAPSSKSTSGGTAALGCLVKDYFPEPVTVSWNSGALTS |
| | (FEAR) | GVHTFPAVLQSSGLYSLSSVVTVPSSSLGTQTYICNVNHKPSNTKVDK |
| | (amino acids positions | RVEPKSCDKTHTCPPCPAPEFEGGPSVFLFPPKPKDTLMISRTPEVTCV |
| | 118-447 according to | VVAVSHEDPEVKFNWYVDGVEVHNAKTKPREEQYNSTYRVVSVLTV |
| | EU numbering) | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSRLTVDKSRWQQGNVFSCSVMHEALHNHYTQKSLSLSPG |
| 21 | IgG1 CH3 region | GQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALH |
| | | NHYTQKSLSLSPG |
| 22 | Constant region | GQPKAAPSVTLFPPSSEELQANKATLVCLISDFYPGAVTVAWKADSSP |
| | human lambda LC | VKAGVETTTPSKQSNNKYAASSYLSLTPEQWKSHRSYSCQVTHEGST |
| | | VEKTVAPTECS |
| 23 | Constant region | RTVAAPSVFIFPPSDEQLKSGTASVVCLLNNFYPREAKVQWKVDNAL |
| | human kappa LC | QSGNSQESVTEQDSKDSTYSLSSTLTLSKADYEKHKVYACEVTHQGLS |
| | | SPVTKSFNRGEC |
| 24 | huCD3-LFLEDA-F405L | EVKLVESGGGLVQPGGSLRLSCAASGFTFNTYAMNWVRQAPGKGLE |
| | (FEAL) | WVARIRSKYNNYATYYADSVKDRFTISRDDSKSSLYLQMNNLKTEDTA |
| | heavy chain | MYYCVRHGNFGNSYVSWFAYWGQGTLVTVSSASTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTCPPC |
| | | PAPEFEGGPSVFLFPPKPKDTLMISRTPEVTCVVVAVSHEDPEVKFNW |
| | | YVDGVEVHNAKTKPREEQYNSTYRVVSVLTVLHQDWLNGKEYKCKV |
| | | SNKALPAPIEKTISKAKGQPREPQVYTLPPSREEMTKNQVSLTCLVKGF |
| | | YPSDIAVEWESNGQPENNYKTTPPVLDSDGSFLLYSKLTVDKSRWQQ |
| | | GNVFSCSVMHEALHNHYTQKSLSLSPG |
| 25 | huCD3 VL + CL light | QAVVTQEPSFSVSPGGTVTLTCRSSTGAVTTSNYANWVQQTPGQAF |
| | chain | RGLIGGTNKRAPGVPARFSGSLIGDKAALTITGAQADDESIYFCALWYS |
| | | NLWVFGGGTKLTVLGQPKAAPSVTLFPPSSEELQANKATLVCLISDFY |
| | | PGAVTVAWKADSSPVKAGVETTTPSKQSNNKYAASSYLSLTPEQWKS |
| | | HRSYSCQVTHEGSTVEKTVAPTECS |
| 26 | CD20-7D8-LFLEDA- | EVQLVESGGGLVQPDRSLRLSCAASGFTFHDYAMHWVRQAPGKGLE |
| | K409R (FEAR) | WVSTISWNSGTIGYADSVKGRFTISRDNAKNSLYLQMNSLRAEDTAL |
| | heavy chain | YYCAKDIQYGNYYYGMDVWGQGTTVTVSSASTKGPSVFPLAPSSKST |
| | | SGGTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPCPA |
| | | PEFEGGPSVFLFPPKPKDTLMISRTPEVTCVVVAVSHEDPEVKFNWYV |
| | | DGVEVHNAKTKPREEQYNSTYRVVSVLTVLHQDWLNGKEYKCKVSN |
| | | KALPAPIEKTISKAKGQPREPQVYTLPPSREEMTKNQVSLTCLVKGFYP |
| | | SDIAVEWESNGQPENNYKTTPPVLDSDGSFFLYSRLTVDKSRWQQG |
| | | NVFSCSVMHEALHNHYTQKSLSLSPG |
| 27 | CD20-7D8 VL + CL | EIVLTQSPATLSLSPGERATLSCRASQSVSSYLAWYQQKPGQAPRLLIY |
| | light chain | DASNRATGIPARFSGSGSGTDFTLTISSLEPEDFAVYYCQQRSNWPITF |
| | | GQGTRLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACE |
| | | VTHQGLSSPVTKSFNRGEC |
| 28 | Human CD3 (epsilon) | MQSGTHWRVLGLCLLSVGVWGQDGNEEMGGITQTPYKVSISGTTVI |
| | | LTCPQYPGSEILWQHNDKNIGGDEDDKNIGSDEDHLSLKEFSELEQSG |
| | | YYVCYPRGSKPEDANFYLYLRARVCENCMEMDVMSVATIVIVDICITG |
| | | GLLLLVYYWSKNRKAKAKPVTRGAGAGGRQRGQNKERPPPVPNPDY |
| | | EPIRKGQRDLYSGLNQRRI |
| 29 | Human CD20 | MTTPRNSVNGTFPAEPMKGPIAMQSGPKPLFRRMSSLVGPTQSFFM |
| | | RESKTLGAVQIMNGLFHIALGGLLMIPAGIYAPICVTVWYPLWGGIM |
| | | YIISGSLLAATEKNSRKCLVKGKMIMNSLSLFAAISGMILSIMDILNIKIS |
| | | HFLKMESLNFIRAHTPYINIYNCEPANPSEKNSPSTQYCYSIQSLFLGILS |
| | | VMLIFAFFQELVIAGIVENEWKRTCSRPKSNIVLLSAEEKKEQTIEIKEEV |
| | | VGLTETSSQPKNEEDIEIIPIQEEEEEETETNFPEPPQDQESSPIENDSSP |

Bold and underlined are F; E; A; L and R, corresponding to positions 234 and 235; 265; 405 and 409, respectively, said positions being in accordance with EU-numbering. In variable regions, said CDR regions that were annotated in accordance with IMGT definitions are underlined.

1. A method of treating Diffuse large B-cell lymphoma (DLBCL) in a human subject, the method comprising administering to the subject a bispecific antibody and an effective amount of lenalidomide and optionally, an effective amount of ibrutinib, wherein the bispecific antibody comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
wherein the bispecific antibody is administered at a dose of 24 mg or 48 mg, and wherein, lenalidomide, the bispecific antibody and optionally ibrutinib are administered in 28-day cycles.

2. The method of claim 1, wherein the bispecific antibody is administered at a dose of 24 mg.

3. The method of claim 1, wherein the bispecific antibody is administered at a dose of 48 mg.

4. The method of any one of claims 1-3, wherein the bispecific antibody is administered once every week (weekly administration).

5. The method of claim 4, wherein the weekly administration of 24 mg or 48 mg is performed for 2.5 28-day cycles.

6. The method of claim 4 or 5, wherein after the weekly administration, the bispecific antibody is administered once every four weeks, such as in 28-day cycles, on day 1 of each 28-day cycle.

7. The method of claim 6, wherein the administration once every four weeks is performed for at least eight 28-day cycles, such as eight 28-day cycles or nine 28-day cycles.

8. The method of claim 6, wherein the administration once every four weeks is performed for at least twenty 28-day cycles, such as twenty 28-day cycles or twenty one 28-day cycles.

9. The method of any one of claims 4-8, wherein prior to the weekly administration of 24 mg or 48 mg, a priming dose of the bispecific antibody is administered in cycle 1 of the 28-day cycles.

10. The method of claim 9, wherein the priming dose is administered two weeks prior to administering the first weekly dose of 24 mg or 48 mg.

11. The method of claim 9 or 10, wherein the priming dose is 0.16 mg.

12. The method of any one of claims 9-11, wherein after administering the priming dose and prior to administering the first weekly dose of 24 mg or 48 mg, an intermediate dose of the bispecific antibody is administered.

13. The method of claim 12, wherein the priming dose is administered on day 1 and the intermediate dose is administered on day 8 before the first weekly dose of 24 mg or 48 mg on days 15 and 22 of cycle 1.

14. The method of claim 12 or 13, wherein the intermediate dose is 0.8 mg.

15. The method of any one of claims 1-14, wherein lenalidomide is administered once a day from day 1 to day 21 of the 28-day cycles.

16. The method of any one of claims 1-15, wherein lenalidomide is administered from cycle 1 to cycle 12 of the 28-day cycles.

17. The method of any one of claims 1-15, wherein lenalidomide is administered from cycle 1 to cycle 24 of the 28-day cycles.

18. The method of any one of claims 1-17, wherein lenalidomide is administered at a dose of 20 to 30 mg, such as 25 mg.

19. The method of any one of claims 1-17, wherein lenalidomide is administered at a dose of 20 to 30 mg in cycle 1 to cycle 12 of the 28-day cycles.

20. The method of any one of claims 1-17, wherein lenalidomide is administered at a dose of 25 mg in cycle 1 to cycle 12 of the 28-day cycles.

21. The method of any one of claims 1-14, wherein lenalidomide is administered at a dose of 10 to 25 mg, such as 25 mg.

22. The method of any one of claims 1-14 and 21, wherein lenalidomide is administered at a dose of 10 to 25 mg in cycle 1 to cycle 24 of the 28-day cycles.

23. The method of any one of claims 1-14 and 21-22, wherein lenalidomide is administered at a dose of 20 mg in cycle 1 to cycle 24 of the 28-day cycles.

24. The method of any one of claims 1-14 and 21-23, wherein ibrutinib is administered once a day from day 1 to day 28 of the 28 day cycles.

25. The method of any one of claims 1-14 and 21-24, wherein ibrutinib is administered from cycle 1 to cycle 24 of the 28 days cycles.

26. The method of any one of claims 1-14 and 21-25, wherein ibrutinib is administered at a dose of 280 to 560 mg, such as 280, 420 or 560 mg.

27. The method of any one of claims 1-14 and 21-25, wherein ibrutinib is administered at a dose of 560 mg in cycle 1 to cycle 24 of the 28 days cycles, or at a dose of 420 mg in cycle 1 to cycle 24 of the 28 days cycles.

28. The method of any one of claims 1, 2, and 4-27, wherein administration is performed in 28-day cycles, and wherein:
(a) the bispecific antibody is administered as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2 and 3, a dose of 24 mg is administered on days 1, 8, 15, and 22;
(iii) in cycle 4 and onwards, a dose of 24 mg is administered on day 1;
(b) lenalidomide is administered on days 1-21 in cycle 1 and onwards, and
(c) ibrutinib is optionally administered on days 1-28 in cycle 1 and onwards.

29. The method of any one of claims 1, 2, and 4-28, wherein administration is performed in 28-day cycles, and wherein:
(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-12, a dose of 24 mg is administered on day 1; and
(b) lenalidomide is administered orally at a dose of 25 mg/day on days 1-21 in cycles 1-12.

30. The method of any one of claims 1, 2, and 4-29, wherein administration is performed in 28-day cycles, and wherein:
(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-24, a dose of 24 mg is administered on day 1;
(b) lenalidomide is administered orally at a dose of 20 mg/day on days 1-21 in cycles 1-24 and
(c) ibrutinib is administered orally at a dose of 560 mg/day on days 1-28 in cycles 1-24.

31. The method of any one of claims 1, 2, and 4-29, wherein administration is performed in 28-day cycles, and wherein:
(a) the bispecific antibody is administered subcutaneously as follows:
(i) in cycle 1, a priming dose of 0.16 mg is administered on day 1, an intermediate dose of 0.8 mg is administered on day 8, and a dose of 24 mg is administered on days 15 and 22;
(ii) in cycles 2-3, a dose of 24 mg is administered on days 1, 8, 15, and 22;
(iii) in cycles 4-24, a dose of 24 mg is administered on day 1;
(b) lenalidomide is administered orally at a dose of 20 mg/day on days 1-21 in cycles 1-24 and
(c) ibrutinib is administered orally at a dose of 420 mg/day on days 1-28 in cycles 1-24.

32. The method of any one of claims 1 and 3-27, wherein administration is performed in 28-day cycles, and wherein:
(a) the bispecific antibody is administered as follows:
(i) in cycle 1, a priming dose of 0.16 mg is administered on day 1, an intermediate dose of 0.8 mg is administered on day 8, and a dose of 48 mg is administered on days 15 and 22;
(ii) in cycles 2 and 3, a dose of 48 mg is administered on days 1, 8, 15, and 22;
(iii) in cycle 3 and onwards, a dose of 48 mg is administered on day 1;
(b) lenalidomide is administered on days 1-21 in cycles 1 and onwards; and
(c) ibrutinib is optionally administered on days 1-28 in cycle 1 and onwards.

33. The method of any one of claims 1, 3-27 and 31, wherein administration is performed in 28-day cycles, and wherein:
(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycles 2 and 3, a dose of 48 mg is administered on days 1, 8, 15, and 22;
(iii) in cycles 4-12, a dose of 48 mg is administered on day 1; and
(b) lenalidomide is administered orally at a dose of 25 mg/day on days 1-21 in cycles 1-12.

34. The method of any one of claims 1, 3-27 and 31-33, wherein administration is performed in 28-day cycles, and wherein:
(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycle 2 and 3, a dose of 48 mg is administered on days 1, 8, 15, and 22;
(iii) in cycles 4-24, a dose of 48 mg is administered on day 1;
(b) lenalidomide is administered orally at a dose of 20 mg/day on days 1-21 in cycles 1-24 and
(c) ibrutinib is administered orally at a dose of 560 mg/day on days 1-28 in cycles 1-24.

35. The method of any one of claims 1, 3-27 and 31-32, wherein administration is performed in 28-day cycles, and wherein:
(a) the bispecific antibody is administered subcutaneously as follows:
(i) in cycle 1, a priming dose of 0.16 mg is administered on day 1, an intermediate dose of 0.8 mg is administered on day 8, and a dose of 48 mg is administered on days 15 and 22;
(ii) in cycle 2 and 3, a dose of 48 mg is administered on days 1, 8, 15, and 22;
(iii) in cycles 4-24, a dose of 48 mg is administered on day 1;
(b) lenalidomide is administered orally at a dose of 20 mg/day on days 1-21 in cycles 1-24 and
(c) ibrutinib is administered orally at a dose of 420 mg/day on days 1-28 in cycles 1-24.

36. The method of any one of claims 1-35, wherein the bispecific antibody is administered subcutaneously.

37. The method of any one of claims 1-36, wherein ibrutinib is administered orally.

38. The method of any one of claims 1-37, wherein lenalidomide is administered orally.

39. The method of any one of claims 1-38, wherein the bispecific antibody, ibrutinib, and lenalidomide are administered sequentially.

40. The method of any one of claims 1-39, wherein the DLBCL is with histologically confirmed CD20+ disease.

41. The method of any one of claims 1-40, wherein the DLBCL is high-grade B cell lymphoma with MYC and BCL-2 and/or BCL-6 translocations (double-hit or triple-hit).

42. The method of any one of claims 1-41, wherein the DLBCL is follicular lymphoma Grade 3B.

43. The method of any one of claims 1-42, wherein the DLBCL is relapsed and/or refractory DLBCL.

44. The method of any one of claims 1-43, wherein the DLBCL has relapsed; i.e. has previously responded to prior therapy but has progressed after said prior therapy, progression having started 6 months or later, after completion of said prior therapy.

45. The method of any one of claims 1-44, wherein the DLBCL is refractory; i.e. has either progressed during prior therapy, has failed to achieve an objective response to prior therapy, or has progressed within 6 months after completion of prior therapy, including maintenance therapy.

46. The method of any one of claims 1-45, wherein the subject has relapsed or refractory disease to at least one prior systemic anti-lymphoma therapy, which contains an anti-CD20 monoclonal antibody.

47. The method of any one of claims 1-46, wherein the DLBCL is not refractory to prior chimeric antigen receptor T cell (CAR-T) therapy.

48. The method of any one of claims 1-46, wherein the subject is not refractory to lenalidomide or ibrutinib.

49. The method of any one of claims 1-48, wherein the subject has received at least 1 prior treatment with an anti-CD20 monoclonal antibody in combination with another systemic therapy.

50. The method of any one of claims 1-49, wherein the subject has received prior CAR-T therapy or is ineligible for or unable to receive CAR-T therapy.

51. The method of any one of claims 1-50, wherein the subject has not had prior treatment with ibrutinib.

52. The method of any one of claims 1-51, wherein:
(i) the first antigen-binding region of the bispecific antibody comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and
(ii) the second antigen-binding region of the bispecific antibody comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.

53. The method of any one of claims 1-52, wherein:
(i) the first antigen-binding region of the bispecific antibody comprises a VH region comprising the amino acid sequence of SEQ ID NO: 6, and the VL region comprising the amino acid sequence of SEQ ID NO: 7; and
(ii) the second antigen-binding region of the bispecific antibody comprises a VH region comprising the amino acid sequence of SEQ ID NO: 13, and the VL region comprising the amino acid sequence of SEQ ID NO: 14.

54. The method of any one of claims 1-53, wherein the first binding arm of the bispecific antibody is derived from a humanized antibody, preferably from a full-length IgG1,λ (lambda) antibody.

55. The method of claim 54, wherein the first binding arm of the bispecific antibody comprises a λ light chain constant region comprising the amino acid sequence set forth in SEQ ID NO: 22.

56. The method of any one of claims 1-55, wherein the second binding arm of the bispecific antibody is derived from a human antibody, preferably from a full-length IgG1,κ (kappa) antibody.

57. The method of claim 56, wherein the second binding arm comprises a κ light chain constant region comprising the amino acid sequence set forth in SEQ ID NO: 23.

58. The method of any one of claims 1-57, wherein the bispecific antibody is a full-length antibody with a human IgG1 constant region.

59. The method of any one of claims 1-58, wherein the bispecific antibody comprises an inert Fc region.

60. The method of any one of claims 1-59, wherein the bispecific antibody comprises a first heavy chain and a second heavy chain, wherein in both the first and second heavy chains, the amino acids in the positions corresponding to positions L234, L235, and D265 in the human IgG1 heavy chain constant region of SEQ ID NO: 15 are F, E, and A, respectively.

61. The method of any one of claims 1-60, wherein the bispecific antibody comprises a first heavy chain and a second heavy chain, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.

62. The method of any one of claims 1-61, wherein the bispecific antibody comprises a first heavy chain and a second heavy chain, wherein
(i) in both the first and second heavy chains, the amino acids in the positions corresponding to positions L234, L235, and D265 in the human IgG1 heavy chain constant region of SEQ ID NO: 15 are F, E, and A, respectively, and
(ii) in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.

63. The method of claim 62, wherein the bispecific antibody comprises heavy chain constant regions comprising the amino acid sequences of SEQ ID NOs: 19 and 20.

64. The method of any one of claims 1-63, wherein the bispecific antibody comprises a heavy chain and a light chain comprising the amino acid sequences set forth in SEQ ID NOs: 24 and 25, respectively, and a heavy chain and a light chain comprising the amino acid sequences set forth in SEQ ID NOs: 26 and 27, respectively.

65. The method of any one of claims 1-64, wherein the bispecific antibody comprises a heavy chain and a light chain consisting of the amino acid sequence of SEQ ID NOs: 24 and 25, respectively, and a heavy chain and a light chain consisting of the amino acid sequence of SEQ ID NOs: 26 and 27, respectively.

66. The method of any one of claims 1-65, wherein the bispecific antibody is epcoritamab, or a biosimilar thereof.


SEQUENCE LISTING (29 sequences)

SEQ ID NO: 1 - protein, 8 aa, synthetic construct
  REGION 1..8: note = CDR sequence
GFTFNTYA

SEQ ID NO: 2 - protein, 10 aa, synthetic construct
  REGION 1..10: note = CDR sequence
IRSKYNNYAT

SEQ ID NO: 3 - protein, 16 aa, synthetic construct
  REGION 1..16: note = CDR sequence
VRHGNFGNSYVSWFAY

SEQ ID NO: 4 - protein, 9 aa, synthetic construct
  REGION 1..9: note = CDR sequence
TGAVTTSNY

SEQ ID NO: 5 - protein, 9 aa, synthetic construct
  REGION 1..9: note = CDR sequence
ALWYSNLWV

SEQ ID NO: 6 - protein, 125 aa, synthetic construct
  REGION 1..125: note = Heavy chain variable region sequence l
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 7 - protein, 109 aa, synthetic construct
  REGION 1..109: note = Light chain variable region sequence
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 8 - protein, 8 aa, synthetic construct
  REGION 1..8: note = CDR sequence
GFTFHDYA

SEQ ID NO: 9 - protein, 8 aa, synthetic construct
  REGION 1..8: note = CDR sequence
ISWNSGTI

SEQ ID NO: 10 - protein, 15 aa, synthetic construct
  REGION 1..15: note = CDR sequence
AKDIQYGNYYYGMDV

SEQ ID NO: 11 - protein, 6 aa, synthetic construct
  REGION 1..6: note = CDR sequence
QSVSSY

SEQ ID NO: 12 - protein, 9 aa, synthetic construct
  REGION 1..9: note = CDR sequence
QQRSNWPIT

SEQ ID NO: 13 - protein, 122 aa, synthetic construct
  REGION 1..122: note = Heavy chain variable region sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 14 - protein, 107 aa, synthetic construct
  REGION 1..107: note = Light chain variable region sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 15 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 18 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 19 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 20 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 21 - protein, 106 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 107 aa, Homo sapiens [sequence omitted]

SEQ ID NO: 24 - protein, 454 aa, synthetic construct
  REGION 1..454: note = Antibody heavy chain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 25 - protein, 215 aa, synthetic construct
  REGION 1..215: note = Antibody light chain
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 451 aa, synthetic construct
  REGION 1..451: note = Antibody heavy chain
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 27 - protein, 214 aa, synthetic construct
  REGION 1..214: note = Antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 29 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP